(19) United States
(12) Patent Application Publication
(10) Pub. No.: US 2025/0177238 A1
(43) Pub. Date: Jun. 5, 2025

(54) SMART ADULT TOY

(71) Applicant: ZIGI CARMEL INITIATIVES & INVESTMENTS LTD, Tel Aviv (IL)

(72) Inventor: Carmel ZIGDON, Tel Aviv (IL)

(21) Appl. No.: 18/726,930

(22) PCT Filed: Jan. 5, 2023
(86) PCT No.: PCT/IL2023/050016

§ 371 Date: Jul. 5, 2024

Related U.S. Application Data
(60) Provisional application No. 63/297,009, filed on Jan. 6, 2022.

(51) Int. Cl. A61H 19/00; A61H 23/02; A61M 31/00

(52) U.S. Cl. CPC A61H 19/44; A61H 23/0254; A61M 31/00; A61H 2201/105; A61H 2201/1246; A61H 2201/5058; A61H 2201/5071; A61H 2201/5082; A61H 2230/065; A61M 2210/1475

(57) ABSTRACT

Some embodiments relate to an adult sex toy is described, comprising: a structure comprising a main head configured to be inserted into a user's cavity; at least one moving member configured to generate selected vibration movement of said structure; at least one sensor located in said main head of said structure and operable for generating sensing data indicative of at least one parameter collected from the user's body cavity; and a controller connectable to said at least one sensor, said controller is configured and operable for receiving and processing said sensing data and for selectively operating said at least one moving member in accordance with one or more conditions determined based on said sensing data.

### CROSS-REFERENCE TO RELATED APPLICATIONS

This application is a national phase filing under 35 C.F.R. § 371 of and claims priority to PCT Patent Application No. PCT/IL2023/050016, filed on Jan. 5, 2023, which claims the priority benefit under 35 U.S.C. § 119 of U.S. Patent Application No. 63/297,009, filed on Jan. 6, 2022, the contents of which are hereby incorporated in their entireties by reference.

### TECHNICAL FIELD

The presently disclosed subject matter relates to adult toys, and specifically relates to adult toys utilizing one or more sensors for controllable operation and liquid administering mechanism

### BACKGROUND

Intimate lifestyle products (interchangeably referred to herein as adult toys, adult sex toys, or “vibrators”) are frequently phallic-shaped objects configured to be inserted into a body cavity, e.g., vagina or anal cavity, to promote organ stimulation through localized vibration from an internal electric motor. There are various known shapes of vibrators and various operation characteristics of such vibrators.

The use of intimate lifestyle products such as vibrators, may require the use of lubricants. These lubricants may increase stimulation or sensation.

U.S. Pat. No. 9,050,240 (whose contents is incorporated herein by reference) offers an improved adult toy. Thus, the '240 patent discloses an adult sex toy containing a self-lubricating system, the adult sex toy being substantially phallus-shaped and configured for insertion into a human body cavity, the adult sex toy comprising: a pipe network: an aperture formed in the adult sex toy, the aperture connected to the pipe network to provide an outlet therefor: a peristaltic pump engaging externally against the pipe network, the peristaltic pump having a rotor assembly configured to positively engage and squeeze a section of the pipe network: a battery supplying electrical energy to the peristaltic pump to power the pump: a hollow core retaining a removable volume-collapsible lubricant cartridge having an inner volume filled, initially, with a dispensable fluid or gel: a tubular piercing member located in the hollow core, the tubular piercing member engaging into the removable volume-collapsible lubricant cartridge to provide a fluid conduit to the pipe network thereby to allow the dispensable fluid or gel to be controllably dispensed from the aperture under operation of the peristaltic pump and wherein the fluid conduit provides an egress path to the inner volume of the removable volume-collapsible lubricant cartridge when the removable volume-collapsible lubricant cartridge is both loaded into the hollow core of the adult sex toy and connected into the pipe network through the tubular piercing member; and a microcontroller coupled to the peristaltic pump to control operation thereof, the microcontroller configured to regulate operation of the peristaltic pump by causing the rotor assembly to selectively spin in order to (i) urge fluid or gel to travel controllably through the pipe network in a direction of the outlet and (ii) control release and delivery of the fluid or the gel from the removable volume-collapsible lubricant cartridge.

It is known that dispending of certain sexual stimulating substances such as liquids containing a sex steroid or a cannabinoid may have a stimulating effect on female sexual desire and receptivity. For example, a scientific article has revealed among others satisfaction in the sexual domains of drive, orgasm and overall sexual experience. Specifically, of the 373 participants, 34.0% (n=127) reported having used marijuana before sexual activity. Most women reported increases in sex drive, improvement in orgasm (see “The Relationship between Marijuana Use Prior to Sex and Sexual Function in Women” Becky K. Lynn, et al. https://www.ncbi.nlm.nih.gov/pmc/articles/PMC6522945/)

With the legalization of both medical and recreational marijuana in some countries and a few US states, its use has become more widely prevalent.

There is a need in the art to provide for a new adult toy configured to dispense cannabinoid substance for stimulating sexual satisfaction in females.

### General Description

The present disclosure provides an adult toy, configured to provide sexual pleasure to a user in accordance with one or more user preferences provided thereby. The adult toy of the present disclosure comprises one or more sensors and a controller configured and operable for monitoring one or more physiological parameters indicative of operation of the adult toy and/or user response to the operation, and adjusting operational parameters in accordance with the one or more physiological parameters.

To this end, the present disclosure provides an adult toy comprising a structure having at least one portion configured to be inserted into a user's body cavity, at least one moving member, at least one sensor and a controller. The controller may comprise one or more processors and memory unit and configured to receive sensing data from the at least one sensor and operate the moving member in accordance with the received sensing data.

The memory unit may store user preference data, e.g., provided by the user at first use or from time to time, and determine according one or more operation patterns. Further, during operation, the controller operates that at least one sensor to collect sensing data, and adjust operation pattern in accordance with sensing data. In some embodiments, the sensing data may provide data indicative of level of sexual stimulation of the user. In some embodiments, variation of operation pattern may be associated with one or more data pieces determined by the user, such as current preference data, desired duration of operation etc.

In some embodiments, the adult toy of the present disclosure further comprises at least one compartment, or chamber, having an inner volume adapted to contain a selected generally-liquid substance, and at least a main conduit configured to deliver the generally-liquid substance to a main liquid/lubricant delivery port. The main delivery port may be located at a distal end of the adult toy structure, typically at a position that is inserted into the user's cavity. The generally-liquid substance may be in liquid or gel form and may include a lubricant and/or one or more active materials selected to enhance sexual pleasure. For example, the generally-liquid substance may contain one or more of: water-based lubricant, oil, cannabinoids extract, CBD, THC or other materials.

Generally, CBD or THC may be used to enhance user pleasure. The euphoric high associated with THC helps to slow things down, and to intensify feelings of sexual pleasure. The CBD oil is known to reduced discomfort, enhance sensations, reduce pain, and ease stress and anxiety. For example, different researches suggest that CBD is able to lower inflammation by affecting the body's activity with endocannabinoid receptors. This includes both chronic and acute inflammation. The CBD oil can handle various medical conditions, such as: vulvodynia, vaginismus, vaginal scarring, pelvic floor dysfunction, uterine fibroids, ovarian cysts, interstitial cystitis. The adult toys disclosed herein, in addition to providing sexual stimulation at the sex organs, may be used for applying selected generally-liquid lubricants at the sex organs to enhance the pleasure and/or relief for which the adult toy is being used by the user. In addition, the adult toys may comprise heating element to heat the generally-liquid substance prior to its delivery for further enhancing the pleasure and/or relief. In some embodiments, heating of the generally-liquid substance may enhance activity and absorption rate of active materials such as CBD and/or THC exiting within the generally-liquid substance.

The at least one compartment may be formed as a chamber for accepting the generally-liquid substance, or for accepting a pre-filled capsule containing the generally-liquid substance.

According to some embodiments, the adult toy may comprise a communication module, e.g., wi-fi, Bluetooth or other types of communication modules configured for communicating with an external electronic device. The adult toy may be configured to receive, via the communication module, user preference data and to store the user preference data. The controller may further utilize stored user preference data for determining operation pattern of the moving member and/or pump, while varying operation pattern in accordance with sensing data during operation.

In some embodiments, where the adult toy utilizes a compartment carrying generally-liquid substance, the adult toy may also comprise a substance indicator indicating data on type of substance used. This may be applied using a sensor configured to identify type of substance containing capsule, or obtaining user input data on type of substance. The controller may determine operation pattern in accordance with the substance used to enhance use of the generally-liquid substance for improving stimulation of the user. Further, the controller may operate in accordance to sensing data using one or more machine learning techniques for varying operation patterns to enhance user satisfaction. To this end, the controller may operate to identify sexual climax based on sensing data, and may operate to obtain user input indicating sexual climax and/or user satisfaction following operation of the adult toy.

The at least one sensor may be at least one pressure sensor, conductivity sensor, temperature sensor, etc. For example, the at least one sensor may comprise at least one sensor located at distance end of the structure at a location that is typically inserted into the user's body cavity during use. In some preferred embodiments, the adult toy may comprise one or more of the different types of sensors and utilize the respective sensing data for determining operational pattern thereof.

Thus, according to a broad aspect, the present disclosure provides an adult sex toy comprising: a structure comprising a main head configured to be inserted into a user's cavity; at least one moving member configured to generate selected vibration movement of said structure; at least one sensor located in said main head of said structure and operable for generating sensing data indicative of at least one parameter collected from the user's body cavity; and a controller connectable to said at least one sensor, said controller is configured and operable for receiving and processing said sensing data and for selectively operating said at least one moving member in accordance with one or more conditions determined based on said sensing data.

According to some embodiments, the adult sex toy may further comprise a compartment having an inner volume and adapted to contain with a selected generally-liquid substance, a main conduit configured to deliver said generally-liquid substance from said compartment through a main liquid delivery port, and at least one pump adapted for pumping said generally liquid substance through said main conduit, said controller is further configured to operate said at least one pump for delivering said generally-liquid substance through said main liquid delivery port in accordance with one or more conditions determined based on said sensing data.

According to some embodiments, the adult sex toy may further comprise at least one auxiliary conduit configured to deliver said generally-liquid substance from the compartment through an auxiliary delivery port distant from the main liquid delivery port; said at least one pump is further adapted for pumping said generally-liquid substance through said auxiliary conduit and through said main conduit.

According to some embodiments, the at least one pump comprises a first peristaltic pump configured to pump the generally-liquid substance from said compartment through the main conduit.

According to some embodiments, the at least one pump comprises a second peristaltic pump configured to pump the generally-liquid substance from said compartment through the auxiliary conduit.

According to some embodiments, the auxiliary delivery port is associated with an auxiliary head port of the adult sex toy configured for removably attaching thereto one or more auxiliary heads, each auxiliary head being configured to perform one or more operations associated with a clitoris of a user of the adult toy.

According to some embodiments, the auxiliary delivery port is configured for attaching thereto a lubricant port extension element of the corresponding auxiliary head for delivering therethrough the generally liquid substance to the clitoris.

According to some embodiments, the moving member may further comprise at least one motor, said controller is further connectable to said motor for selectively controlling one or more parameters of operation of said at least one motor in accordance with said sensing data.

According to some embodiments, the controller comprises at least one processor and memory and is configured to received and store input data indicative of one or more predefined user preference modes; said controller is further configured for processing said sensing data in accordance with said one or more user preference modes.

According to some embodiments, the at least one sensor comprises at least one pressure sensor positioned near top end of said moving member and operable for providing sensing data indicative of time-varying pressure applied thereon, said data on time-varying pressure being indicative of use and user stimulation level.

According to some embodiments, the adult sex toy may further comprise one or more additional pressure sensors located at one or more positions along said moving member, said sensing data comprises data indicative of time-varying pressure along said moving member.

According to some embodiments, the at least one sensor comprises at least one temperature sensor positioned near top end of said moving member, said sensing data comprises temperature variation data.

According to some embodiments, the adult sex toy may further comprise at least one additional temperature sensor positioned at distal end of said moving member, such that said additional temperature sensor is external to said user's body cavity when said moving member is inserted into the user's body cavity, said additional temperature sensor provides sensing data indicative of external temperature, said sensing data thereby comprises data indicative of temperature difference between said at least one temperature sensor and said at least one additional temperature sensor.

According to some embodiments, the controller is configured for processing said sensing data to determine temperature difference in data obtained from said at least one temperature sensor and said at least one additional temperature sensor, said controller is configured to use data on said temperature difference and determine data on presence of said moving member within said user's body cavity.

According to some embodiments, the controller is configured to determine environment status indicative of presence of said moving member within said user's body cavity based on temperature data collected by the at least one sensor.

According to some embodiments, the at least one sensor comprises at least one conductivity sensor, said controller is configured for processing conductivity data ins aid sensing data to determining one or more parameters comprising at least data on liquid material composition around said moving member.

According to some embodiments, the controller is configured for processing said sensing data to determining data indicative of heart rate of a user based on periodic variations in said sensing data in the range of 50 to 200 beats per minute, and for utilizing said heart rate data to determine estimated stimulation level of the user.

According to some embodiments, the controller comprises pre-stored user preference data, and wherein said controller is configured and operable for receiving and processing sensing data from the at least one sensor in accordance with user preference data and determining user stimulation level during operation.

According to some embodiments, the user preference data comprises data indicative of user preferences for adult toy operation during sexual climax.

According to some embodiments, the controller is configured and operable for correlating sensing data collected from one or more sensors collecting respectively one or more different types of sensing data for determining user stimulation level during operation.

According to some embodiments, the generally liquid substance comprises a lubricant. According to some embodiments, the generally-liquid substance may comprise at least one active material. According to some embodiments, the generally-liquid substance may comprise one or more active elements comprising THC or CBD.

According to some embodiments, the controller is configured to receive input data indicative of presence of one or more active elements in said generally liquid substance and to selectively operate in at least first or second operation modes selected in accordance with presence of active elements in said generally liquid substance. To this end, the adult sex toy may comprise a capsule reader configured for reading capsule marking indicating type of substance within different capsules and determine operation pattern in accordance with one or more active materials present or lacking from the generally-liquid substance in the capsule.

According to some embodiments, the controller comprises stored data indicative of one or more operational programs preselected by a user, and for processing said sensing data to determine one or more operational parameters of said at least one moving member and said at least one pump in accordance with a selected operational program in said stored data.

According to some embodiments, the one or more operational programs comprise data on at least first short duration and a second longer duration, and wherein in response to selection of said second duration said controller is configured to perform repeatedly: determining if, based on said sensing data, the stimulation intensity will lead to satisfaction faster or slower than said desired duration and in response thereto modify at least one of (i) a moving command to modify at least one of moving magnitude and speed of said moving member; (ii) a dispending command to modify the released dosage of said generally liquid substance from the compartment.

According to some embodiments, the adult sex toy may further comprise at least one battery providing electrical power for operation of said adult sex toy.

According to some embodiments, the controller comprises a communication module configured for communicating with a selected electronic device for input data of user preference and operational modes.

According to some embodiments, the communication module is configured to use Bluetooth communication protocol.

According to one other broad aspect, the present disclosure provides a method for controlling a battery-operated adult sex toy configured according to any one of claims 1 to 25, the method comprising:
(a) using at least one pre stored user preference data comprising at least data indicative of desired duration of operation of the toy until user's sexual stimulation arrives to satisfaction;
(b) obtaining, from said at least one sensor, sensing data indicative of one or more physiologic originated signals from the user's body; said physiologic originated signals being informative of the sexually stimulation intensity of the user while using the toy;
(c) processing sensing data to determine respective physiologic originated signals and determining at least one of: (i) a moving command for controlled modification of the magnitude of move of the moving member, and (ii) a dispensing command, for controlled dispensing of the stimulating substance from the cartridge by said dispensing control member,
thereby substantially matching the actual duration of operation of the toy with the desired input duration of operation, until satisfaction is arrived.

According to one additional broad aspect, the present disclosure provides a method for operating an adult sex toy, the method comprising:
obtaining user preference data, and storing the suer preference data in a memory;
responding to user initiation instruction for operating the adult sex toy;
operating one or more moving members for stimulating one or more selected user body parts;
collecting sensing data comprising at least one of: pressure variations, heart rate, humidity, and temperature, from vicinity of the one or more selected user body parts;
varying operation of the one or more moving members in accordance with said sensing data and respective user preference data to bring the user to satisfaction;
updating stored user preferences based on sensing data for additional use sessions.

According to some embodiments, the method may further comprise processing the sensing data and determining user stimulation level accordingly.

According to some embodiments, said processing user stimulation level comprises determining a combination of time correlated of one or more of pressure variation frequency within range between 0.5 Hz and 2 Hz, heart rate increase within the range 10%-25% of initial heart rate, temperature increase between 0.5° C. and 3° C., and humidity increase that is not related to lubricant dispensing.

According to some additional aspect, the present disclosure provides a computer readable program code, embedded on a computer readable medium (e.g., non-transitory computer readable medium such as HDD, CD, etc.) and comprising code that when executed by one or more processor cause the processor to perform method actions listed herein.

According to one further broad aspect, the present disclosure further provides a system, method, and computer program device configured for operating an adult sex toy in accordance with pre-stored user preference data and sensing data collected by one or more sensors thereof to provide sexual stimulation to a user, and further update scores of one or more operation actions (e.g., vibration frequency, strength, liquid dispensing, etc.) in accordance with user response to update the stored user preference data for further use sessions.

### BRIEF DESCRIPTION OF THE DRAWINGS

In order to understand the presently disclosed subject matter and to see how it can be carried out in practice, embodiments will be described, by way of non-limiting examples, with reference to the accompanying drawings, in which:

FIGS. 1A-C illustrate schematically an adult toy (by this particular embodiments a vibrator) according to certain embodiments of the presently disclosed subject matter;

FIG. 2 illustrates a functional block diagram of a controller in accordance with certain embodiments of the presently disclosed subject matter;

FIG. 3 illustrates a generalized flow-chart of a sequence of operations performed by a controller, in accordance with certain embodiments of the presently disclosed subject matter.

FIGS. 4A to 4H illustrate structure of an adult toy according to some embodiments of the present disclosure, FIG. 4A exemplifies external structure of the adult toy, FIG. 4B shows a cross-section of the adult toy, FIG. 4C exemplifies structure with removed components for illustration purposes, FIGS. 4D and 4E show additional perspectives of the adult toy, FIG. 4F illustrates an additional perspective of the adult toy, FIG. 4G shows an enlarged view of a cross-section taken along line B-B in FIG. 4F, and FIG. 4H shows another view of the adult toy according to some embodiments of the present disclosure;

FIG. 5 exemplifies user interface via an associated electronic device according to some embodiments of the present disclosure;

FIG. 6 exemplifies method of operation of an adult toy according to some embodiments of the present disclosure;

FIG. 7 exemplifies processing of temperature data according to some embodiments of the present disclosure;

FIG. 8 exemplifies processing of conductivity data according to some embodiments of the present disclosure; and

FIG. 9 exemplifies determining user stimulation level according to some embodiments of the present disclosure.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, components and circuits have not been described in detail so as not to obscure the presently disclosed subject matter.

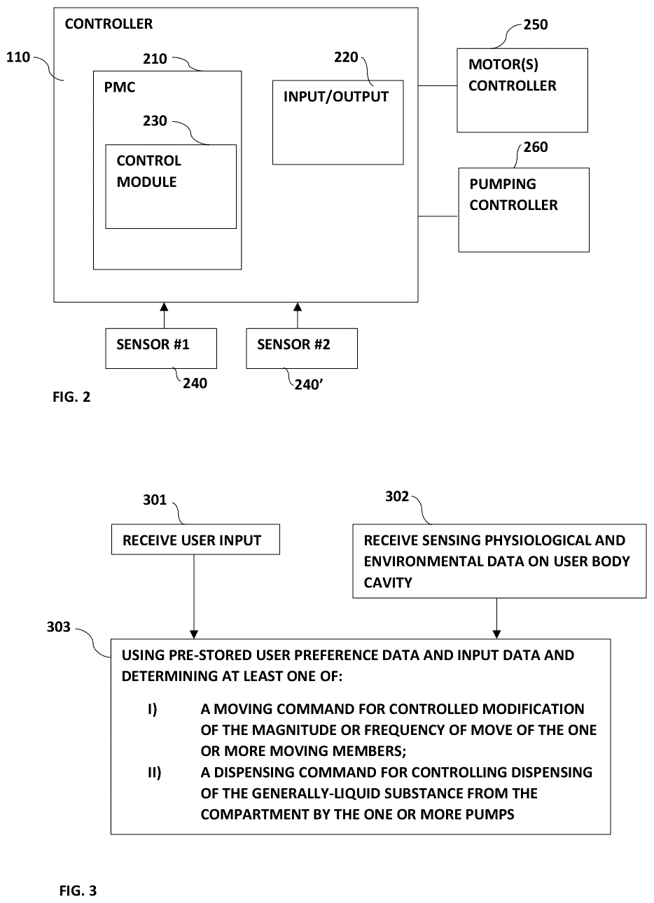

Unless specifically stated otherwise, as apparent from the following discussions, it is appreciated that throughout the specification discussions utilizing terms such as “processing”, “determining”, or the like, refer to the action(s) and/or process(es) of a computer that manipulate and/or transform data into other data, said data represented as physical, such as electronic, quantities and/or said data representing the physical objects. The term “computer” (including controller) should be expansively construed to cover any kind of hardware-based electronic device with data processing capabilities including controller that comprises Processor Memory Circuitry (PMC) shown by way of non-limiting example, in FIG. 2 disclosed in the present application.

The terms “non-transitory memory” and “non-transitory storage medium” used herein should be expansively construed to cover any volatile or non-volatile computer memory suitable to the presently disclosed subject matter.

The operations in accordance with the teachings herein may be performed by a computer specially constructed for the desired purposes or by a general-purpose computer specially configured for the desired purpose by a computer program stored in a non-transitory computer-readable storage medium.

Embodiments of the presently disclosed subject matter are not described with reference to any particular programming language. It will be appreciated that a variety of programming languages may be used to implement the teachings of the presently disclosed subject matter as described herein.

Note that throughout the description whenever reference is made to a given term that represents data or information, it may be construes, if applicable, to embrace also data informative of the term including but not limited to different representations and formats, such as (but not limited to) physiologic acquired signals being informative of the sexually stimulation intensity of the user while using the toy.

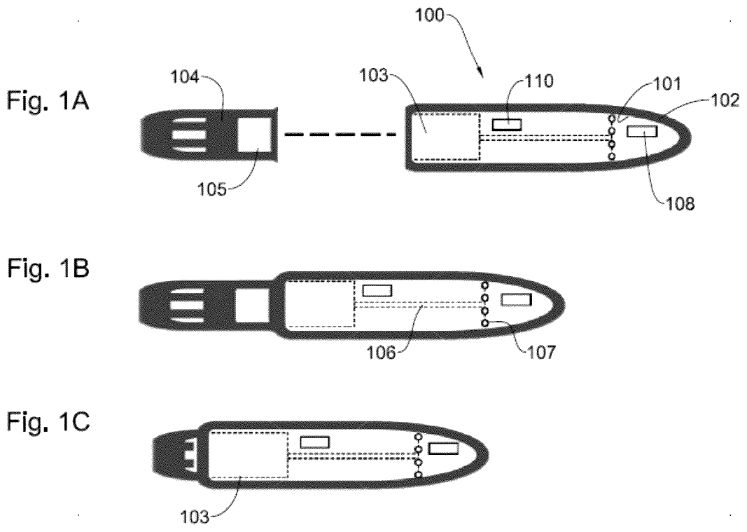

Before moving on, it should be noted that the structure of an adult sex toy such as a vibrator is generally known per se and therefore will only be outlined herein. For example, essential structural and operational details such as pumps, motors ducts, housing, handle, selection buttons (for various modes of operations), battery compartment, wiring display and other elements that are familiar to a person versed in the art (in terms of structure and manner of operation) are not described herein. By way of non-limiting example, a more comprehensive structure and manner of operations is described in U.S. Pat. No. 9,050,240. The presently disclosed subject matter is not bound by any structure of adult toy (e.g., vibrator, dildo) and particularly not by the one depicted in FIGS. 1A-1C herein and/or described with reference to the specified '240 patent.

In this connection FIGS. 1A-1C exemplify an adult sex toy 100, also referred to herein as vibrator. The adult sex toy 100 exemplified herein includes a structure 102, having a generally phallic shape and configured to be inserted into a user's cavity (typically vagina or anus), a moving member 101 and controller 110. The moving member may be operated using a motor 108 for generating vibrations in accordance with a selected pattern. The adult sex toy further includes a compartment 103 configured to contain a generally-liquid substance such as a lubricant and a main conduit 106 configured to direct the generally-liquid substance from the compartment 103 to exit through a main liquid/lubricant delivery port 107.

Generally, the example of FIGS. 1A-1C, illustrating schematically an adult toy 100 according to certain embodiments of the presently disclosed subject matter utilizing one moving member 101 and a generally phallic structure 102 configured to be inserted into a user's body cavity (typically a vagina or anus—not shown). It should be noted that for simplicity only one moving member 101 is shown, there may be different and additional moving part(s) of the toy 100 that move (e.g., vibrate and/or rotate), all as known per se. For instance, in accordance with various embodiments only the tip member at leading edge (not shown) is configured to move. By the example of FIG. 1A, the moving member is activated by a vibrator module e.g., a motor is indicated as 108. Further, in some examples as described further below, the adult toy 100 may include an auxiliary unit configured to stimulate external organs of the user, such as the clitoris, and may include additional moving members and respective motors.

Further, as shown in FIG. 1C, the vibrator 100 may also include a compartment 103 for accommodating a generally-liquid substance. The compartment may be formed as an internal chamber for storing the generally liquid substance or configured to accept one or more removable cartridges/capsules 104 having an inner volume 105 that can be filled with the generally-liquid substance. The generally-liquid substance may be a lubricant and may also include a dispensable sexually stimulating substance (e.g. liquid), such as cannabinoid extract. Typical yet not exclusive examples being CBD or CBD oil and/or THC. The cartridge 104 is fitted to the container 103 as can be seen e.g., in FIG. 1C. enabling selective use of the generally-liquid substance based on selected cartridge 104. The cartridges may be provided with specific marking in the form of structural patterns, computer readable code, RFID etc. providing cartridge data to the controller 110. The controller may be configured to receive cartridge identity data and determine operational pattern accordingly. Such operational pattern may include level of pumping and amount of the generally-liquid substance to be applied at selected times.

It should be noted that the chemical formulation of the generally-liquid substance, and/or biological operation thereof may vary. For simplicity, the description below focuses on stimulated liquid or cannabinoid extract, but those versed in the art will appreciate that the presently disclosed subject matter is not bound by these examples.

As shown, the adult sex toy 100 may include a liquid conveyer, conduit 106, providing path for conveying the generally-liquid substance from the compartment 103 or cartridge 104 to exit through the one or more delivery ports 107 into the user's body cavity. The conduit 106 may be associated with a pump, operated by the controller 110 for selectively providing the generally-liquid substance in accordance with operation pattern and/or sensing data, Typically, the generally-liquid substance may be sued as lubricant for reducing friction of the structure 102 with inner body cavities. To this end, a typical operation pattern may include initial discharge of selected amount of the generally-liquid substance. It should be noted that the illustration of FIGS. 1A to 1C show a single conduit 106 additional one or more conduits may be used in accordance with structural design of the adult toy 100.

It should also be noted that FIGS. 1A-1C illustrate the cartridge 105 being in extracted position in FIG. 1A, positioned within the compartment 103, in operational position in FIG. 1C (and at interim stage in FIG. 1B). It should be noted that the specific configuration and extraction/inserting technique of the cartridge may vary and is of course not bound to compartment and or cartridge structure. Other non limiting examples are shown in the specified '240 patent.

The adult toy 100 also includes a dispending control member (e.g. including at least one pump), which is not specifically shown here. The pump is being responsive to a dispensing command from the controller 110 for controlled dispensing of the generally-liquid substance from the cartridge 105 to the user cavity (by this particular embodiment through the conduit to the apertures), as explained in greater details below. Further, the adult toy 100 may include a piercing arrangement for piercing/opening the cartridge and extracting or allowing flow of the generally-liquid substance in accordance with determined operation pattern.

As indicated above, the moving member 101 may be operated by a motor 108. The motor may be configured to operate in selected operation speed and duration. Generally, the controller 110 may include a moving control module. The moving control module operates to transmit operational instructions to the motor in accordance with operation pattern. The operation instructions may typically determine motor speed, duration, pulsating operation, vibration intensity, vibration frequency etc. Selection of these parameters along a selected duration describes an operation pattern of the adult toy 100, typically, as described in more details further below, the controller may determine operation pattern in accordance with pre-stored user preference data and may adjust and change the operation pattern in accordance with sensing data obtained from the one or more sensors.

As further mentioned above, the adult toy 100 includes at least one sensor (not specifically shown). The at least one sensor is configured to acquire sensing data indicative of environmental and/or physiologic signals from the user's body, and to transmit the collected sensing data to the controller 110. The sensing data may include data indicative of the sexually stimulation intensity of the user while using the toy, as well as additional data that may indicate on user satisfaction, moisturizing etc. Typically, the adult toy 100 may include an arrangement of sensors providing sensing data in a plurality of sensing data channels. For examples, the arrangement of sensors may include one or more sensors including: pressure sensors, temperature sensor, conductivity sensor, optical heart rate sensor, and optionally additional sensors. The sensors are generally operated at a selected sampling rate to provide sensing data to the controller 110. The controller operates for processing the sensing data, e.g., using one or more processing techniques such as Fourier transform, correlation detection, machine learning techniques etc. The controller may operate to determine level of sexual stimulation and determine an operation pattern that will provide sexual satisfaction in accordance with user's preferences (e.g., regarding time, duration etc.). Additionally, the controller may process the sensing data for determining data on moisturization status of the user's cavity and operate the pump for providing added amount of the generally-liquid substance for lubricating the region within the user's cavity.

For example, the controller 110 may process the sensing data for determining heart rate, temperature and pressure variations for determining level of sexual satisfaction, progressing toward climax, and sexual climax/orgasm. The controller may record and store data indicative of the sensing data and user experience data for enhancing user satisfaction in additional use sessions.

Additionally, as indicated above, the adult toy may utilize a user interface for operation and for providing user input data. To this end, the controller generally includes an I/O interface (e.g. a switch or a touch panel) as well as communication module for receiving user preference data, user input and allowing a user to observe operation data. For example, the adult toy may be in wireless communication with one or more electronic devices for receiving and providing user communication data and/or operational data. In some embodiments, the adult toy may request user preference data, e.g., in a set of questions, at first use and periodically to update suer preferences. In some further embodiments, the adult toy may be responsive to user indications for changing/adjusting operational patterns. In some embodiments, the adult toy may be responsive to user input data on satisfaction level, e.g., did the suer reach an orgasm, and/or receiving general operation feedback.

The controller 110 may generally be in the form of a computer processor associated with a memory unit. The computer processor may be operable for executing one or more sets of computer readable instructions in accordance with user input and sensing data to determine operation pattern of the adult toy 100. The controller 110 may generally be configured for determining at least one of: (i) a moving command for controlled modification of frequency and/or magnitude of move of the moving member 101, and (ii) a dispensing command, for controlled dispensing of the generally-liquid substance from the cartridge 105. Dispensing commands may be used in accordance with data on the generally-liquid substance. For example, when operated with general lubricant substance, the dispensing commands may be controlled by data on moisturization and level of lubricant in the user's body cavity. For example, the controller may operate to determine moving member operation pattern and dispensing generally-liquid substance to substantially match the actual duration of operation of the toy with data on desired input duration of operation, until satisfaction is arrived.

Generally, the controller 110 may be placed within housing of the adult toy 100, i.e., within the structure 102 thereof. However, it should be noted that the controller 110 may be placed in an external housing connected by wired or wireless communication with the elements of the adult toy within the structure 102. The adult toy may be controlled using one or more user interface elements (e.g., buttons) on the structure 102 itself, using remote connected vie a connected electronic device (e.g., smartphone), and/or using a remote control unit. Accordingly, the controller 110 may reside locally or distributed. In the latter case, the controller 110 may include a remote member associated with end-user device, such as but not limited to cellular phone. For the remote member, the computational modules may reside at the end-user unit and/or distributed partially or wholly at remote server(s) including but not limited to a cloud.

Reference is made to FIG. 2 illustrating a functional block diagram of the controller 110 in accordance with certain embodiments of the present disclosure. The controller 110 is typically a computer-based system capable of controlling at least the one or more motors operating the moving member 101, and in some embodiments, also controlling pump operation for dispensing the generally-liquid substance.

As shown, the controller 110 includes a processor and memory circuitry (PMC) 210 operatively coupled to a hardware-based I/O interface 220 for receiving user inputs such as desired duration of operation (e.g. short or long), indication of satisfaction, and other user preference data, The I/O interface is typically also configured to receive sensing data indicative of physiological 240 and/or environmental 240′ characteristics collected by the one or more sensors. The PMC 210 generally operates for processing the collected sensing data, e.g., for determining heart rate data, pressure variation data, data on presence of lubrication and liquids in the body cavity etc., for generating environmental and physiological sensing data out of the collected raw sensing data. The PMC 210 and is further configured or transmitting commands to the motor(s) controller 250 for controlling operation of the one or more motors operating the moving member 101 and is some embodiments to send commands to the pumping controller 260 for operating the one or more pumps for dispensing the generally-liquid substance. PMC 210 is configured to provide all processing necessary for operating the adult toy system 100 as further detailed below and comprises a processor (not shown separately) and a memory (not shown separately). The processor of PMC 210 can be configured to execute several functional modules in accordance with computer-readable instructions implemented on a non-transitory computer-readable memory comprised in the PMC. Such functional modules are referred to hereinafter as comprised in the PMC. As explained above the controller may reside locally, i.e., fitted within structure 102 of the adult toy 100 or partially or wholly remotely being configured to operate the adult toy by wired or wireless communication. The memory of the PMC 210 may generally store operational instructions to be executed by the processor, as well as operational and user data indicative of user input data, user preferences etc. Typically, the PMC 210 may operate to store data indicative of user response and satisfaction input enabling adjustment of operation of the adult toy 100 in response to previous user experience.

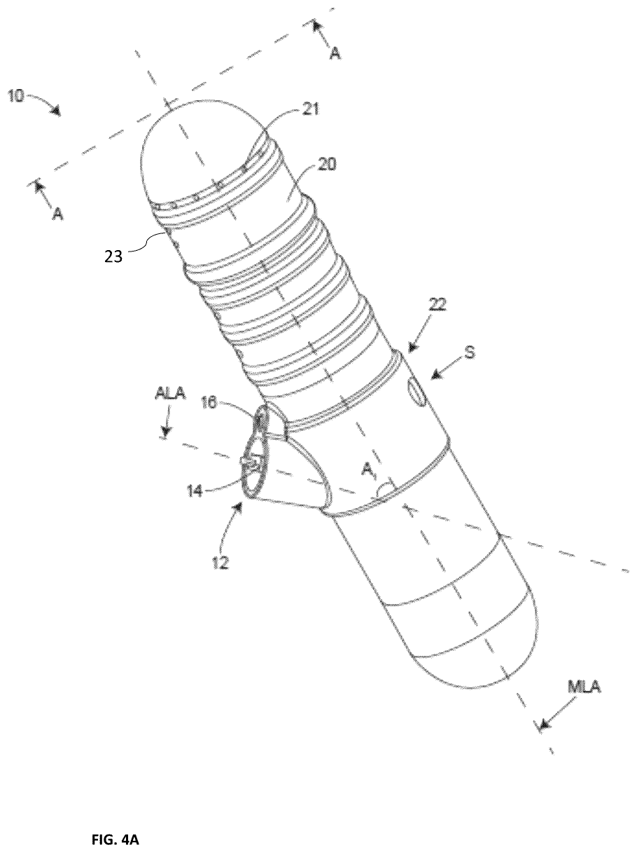

Operation of controller 110, PMC 210 and the functional modules therein are further detailed with reference to FIG. 3. FIG. 3 illustrates a general scheme of operation of the adult toy according to some embodiments of the present disclosure. As shown, the PMC 210 receiving input data including user input data 310 indicative of current user preferences, instructions for current and/or general operation of the system. Additionally, during operation, the PMC 210 received sensing data 302 including data collected by the one or more sensors. The PMC 210 operates for processing the received data 303 and determining operation of the one or more motors and/or the one or more pumps in accordance with the input data, and optionally in accordance with pre-stored data indicative of past user experience and general user preferences. It should be noted that the general scheme illustrated herein is an example and the present disclosure is not bound by the specified modules and the various sequences operations described with reference to various embodiments herein may be implemented by modified or different modules that those described with reference to FIG. 2.

referring back to FIG. 3 the flow-chart of a sequence of operations performed by a controller is illustrated, in accordance with certain embodiments of the presently disclosed subject matter. as indicated, the controller may be configured to receive user's input (301), and to be responsive to sensing data indicative of acquired physiologic and/or environmental signals (302) originated from the user's body cavity, for determining at least one of: (i) moving commands for controlled modification of at least one of magnitude and frequency of move of one or more moving members, and generally selection between the moving members, and (ii) a dispensing command, for controlled dispensing of the generally-liquid substance for stimulating and/or lubricating the user's body cavity or surrounding thereof by operating one or more pumps for delivering the substance through the respective conduit (303). The operational commands determined by the PMC are generated in accordance with user preferences data in order to provide user experience in accordance with user preferences relating to duration of operation, user satisfaction, preferred operation type of the adult toy 100. The PMC 210 may also utilize the sensing data, and optionally user input data, for determining satisfaction level of the user and sexual climax, enabling the adult toy to identify satisfaction and/or climax and store respective physiological parameters and operation scheme in the memory to improve user experience in further operations of the system.

In accordance with certain embodiments of the present disclosure, the one or more input sensors may include one or more sensors for sensing data informative of heart beats of the user, e.g., optical IR sensor. Additional sensors may include conductivity sensor, pressure sensor, temperature sensor (e.g., thermocouple) etc.

In accordance with certain embodiments, in operation, consider the example that the user input being informative of said second (longer) duration. The controller 110 may be configured to perform (possibly through the control module 230) the following, repeatedly:
determine if, based on the input signals, the stimulation intensity is leading to satisfaction faster or slower than said desired duration and in response thereto modify at least one of (i) the moving command for modify the moving (e.g. vibration) magnitude of the moving member (250); (ii) the dispending command to the dispensing control member 260, for modify the released dosage of the stimulating liquid (e.g. the cannabinoid substance) from the cartridge (e.g. 104). In accordance with certain embedment if the first option is selected (short duration), the controller will operate in a default mode of commanding a fixed dosage of stimulating substance as modifying the dosage will have limited effect considering the desired short time duration until satisfaction is arrived.

Moving on with the latter example, consider an embodiment of a first sensor 230 configured to sense data informative of heart beats of the user, and wherein the determination (by the controller) if the sensed signals will lead to satisfaction faster or slower than said desired duration is determined based on the heart rate of the user. By this embodiment the controller will modify the specified commands (i) and/or (ii) based e.g. on the change of sensed heart rate of the user (e.g., change in heart rate). For example, if the heart beat rate increases and the controller determines that the stimulation intensity will lead to satisfaction faster than said desired duration inputted by the user, the controller may issue a command (fed to the control moving member, e.g. a vibrating motor) to reduce the vibration intensity and/or a command to reduce the released dosage of the cannabinoid substance from the cartridge.

In accordance with another embodiment a second sensor 240 (in line or in addition to said first sensor) configured to sense data informative of the vaginal moisture of the user by determining conductivity data thereof, and wherein the determination by the control module 230 if the input signals will lead to satisfaction faster or slower than said desired duration is determined based on the vaginal moisture value (e.g. change of moisture value rate). The operation of the controller for modifying the command is similar to that described with reference to the specified first sensor.

Note that in accordance with other embodiments, the controller may determine the desired command(s) to the moving control member (increase/decrease intensity) and/or dispensing control member (increase decrease dosage) based on input from both sensors.

In accordance with certain embodiments, the controller 200 includes a local controller fitted in said the and a remote controller associated with end-user communication device (not shown) and communicating with the adult toy in a known per se manner e.g. through cellular and/or internet infrastructure. The end-user communication device may be by way of non-limiting example a cellular phone laptop and so forth.

In accordance with various embodiment, the remote controller is configured to receive said use's input, and repeatedly receive data informative of the physiological originated signals, and in response thereto determining at least one of: (i) a moving command for controlled modification of the magnitude of move of the moving member, and (ii) a dispensing command, for controlled dispensing of the stimulating liquid from the cartridge, and transmitting (through the network—not shown) said commands to the local controller for feeding to said moving control member and dispensing control member.

Note that the controller may utilize also non physiological originated data, such as age height and weight which may affect for instance the dosage hat should be dispensed. Note also that if the feeds her health condition which may prescribe to limit the maximal dosage of stimulating substance that can be dispensed, the controller (through appropriate command) will prohibit the release of the substance if it exceeds the allowed quota.

Thus, in accordance with various embodiments, the following advantage is achieved: the actual duration of operation of the toy substantially matches the desired input duration of operation, until user's satisfaction is arrived.

For purpose of illustration only, the following description is provided for an application to enhance sexual arousal.

It is noted that the teachings of the presently disclosed subject matter are not bound by the flow chart illustrated in FIG. 3 or by various embodiments described with reference to the operation of the controller. It is also noted that whilst the flow chart is described with reference to elements of controller (200) of FIG. 2 this is by no means binding, and the operations can be performed by elements other than those described herein. Accordingly in accordance with certain embodiments various computational stages performed by the controller may be deleted and or combined with others, and or executed in different order and/or modified and/or others may be added in addition or instead those illustrated in the drawings.

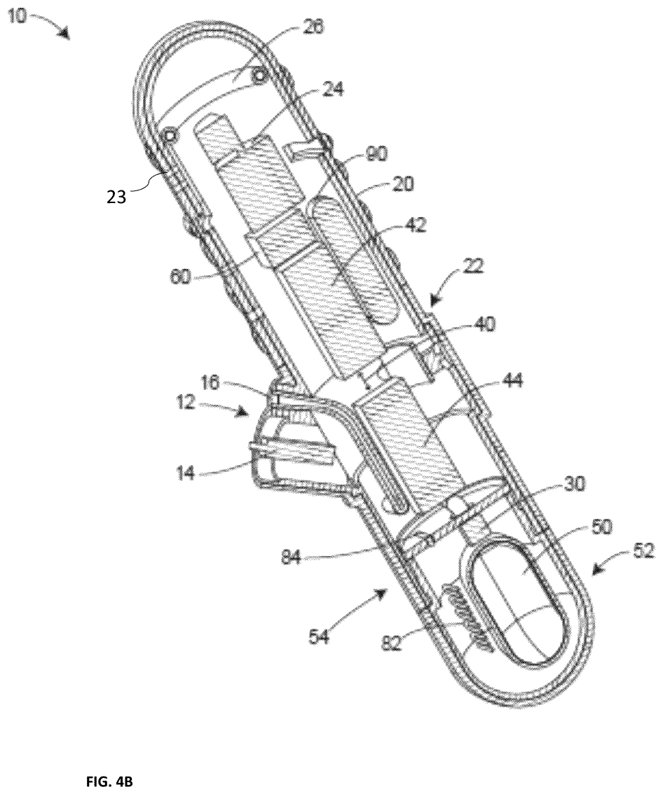

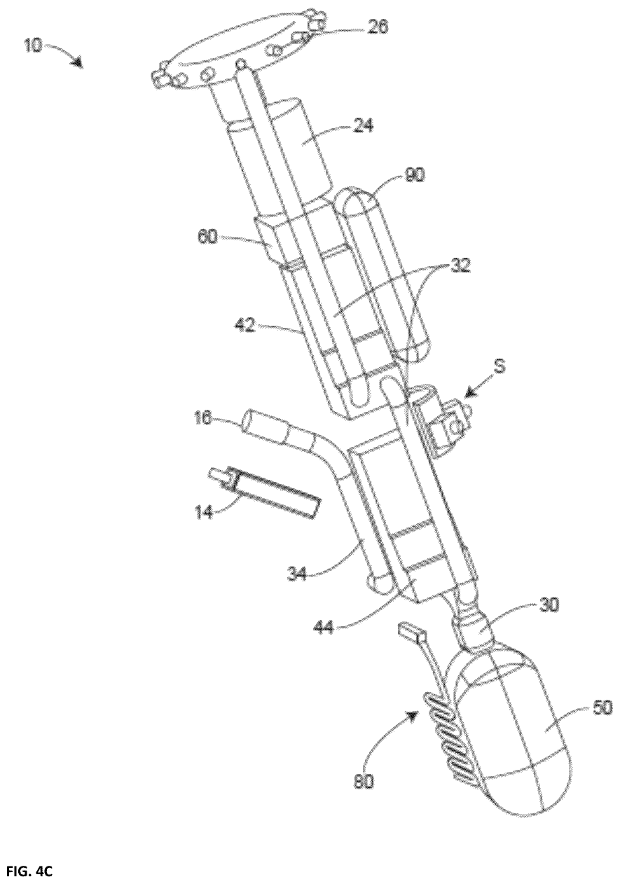

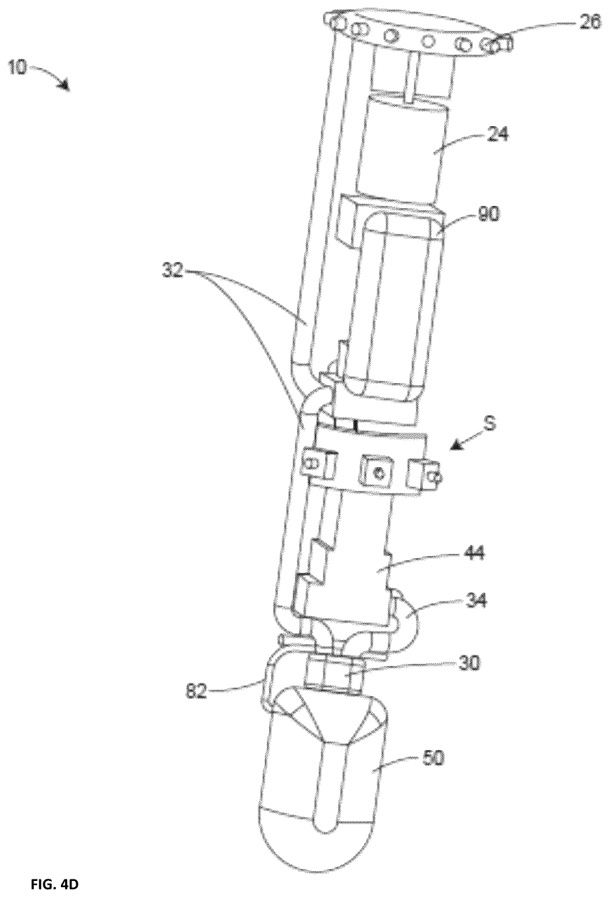

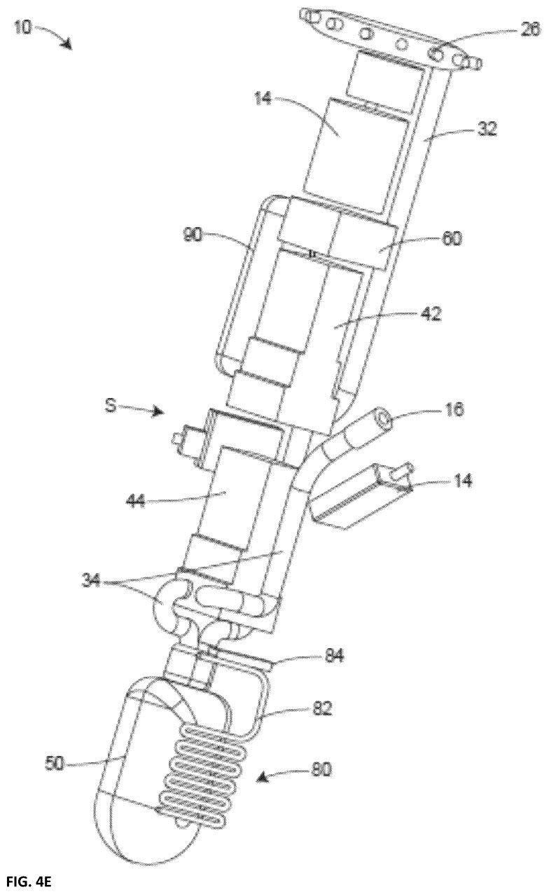

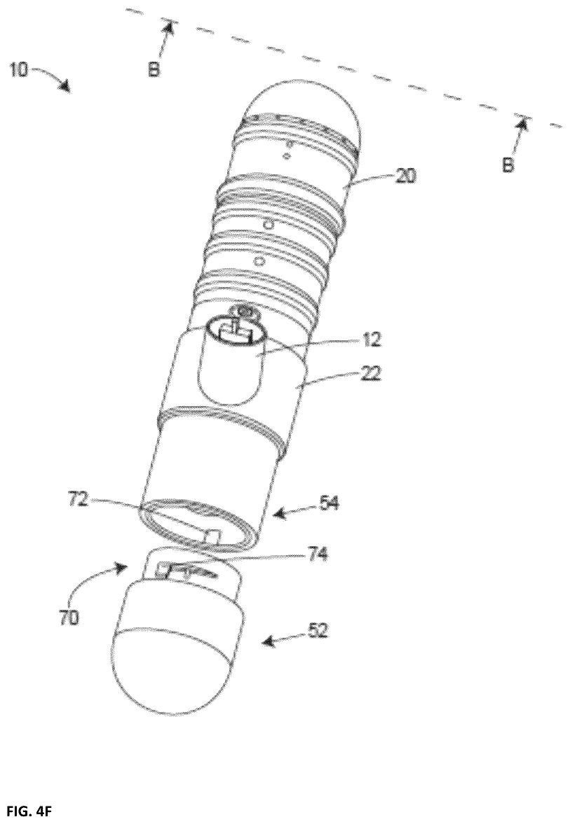

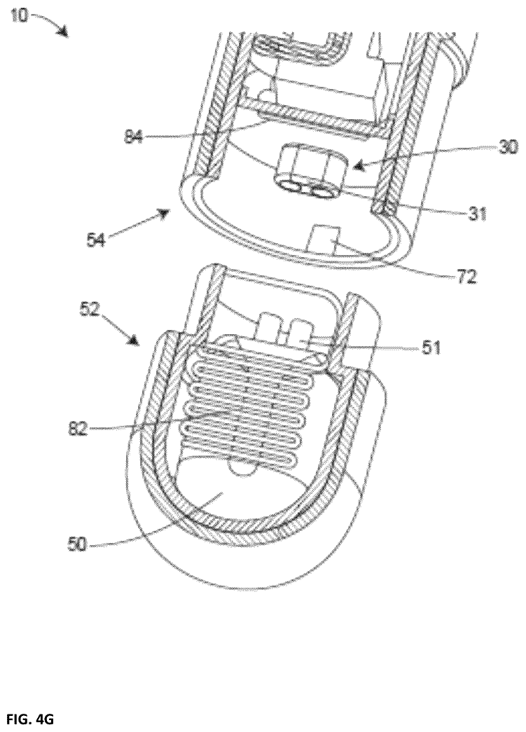

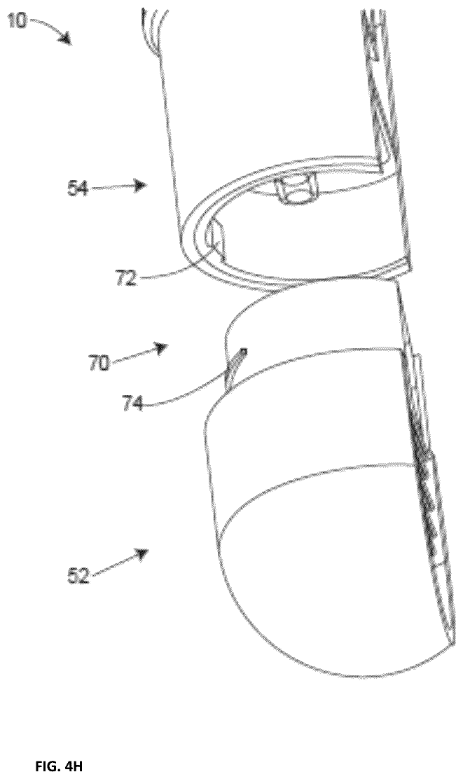

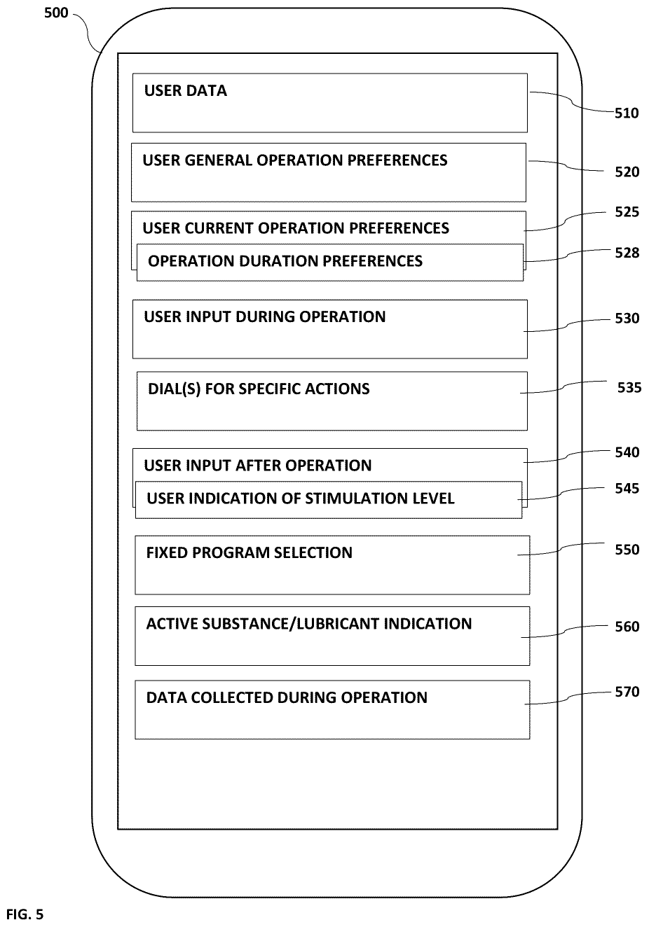

Reference is now made to FIGS. 4A to 4H illustrating an adult toy 10 according to some exemplary embodiments of the present disclosure. As indicated above, the adult toy 10 in general is a sex toy configured for use to stimulate sex organs of a user, e.g., a vagina and clitoris of a female user. In some exemplary configuration, the adult toy 10 may have a head port 12 configured for removably attaching thereto one or more operative heads, each operative head comprising at least one operational unit, described in more details in patent application PCT/IL2022/050783 assigned to the assignee of the present application. The adult toy 10 further has a motor 14 positioned at the head port 12 and configured to be operationally coupled to the operational unit of each of the operative heads for facilitating performance of operation thereof.

In general, the head port 12 is configured for removably attaching thereto the operative heads by an attaching action. The attaching action can be simply pressing the operative head on to the head port 12 along a longitudinal axis thereof. The motor 14 is configured to be operationally coupled to the corresponding operational units during said attaching action. In the illustrated example, the head port 12 extends along its longitudinal axis ALA and is configured to receive the operative heads therealong. The operative head can be pressed on to the head port 12 along the longitudinal axis ALA and attached thereto by for example, a snap attachment. In some examples, the attachment can be by a bayonet twist lock or by a simple frictional attachment by virtue of dimensions of the head port and the operative heads. The motor 14 is configured, i.e., structured, positioned, and oriented so as to be operationally coupled to the operational unit of the operative head when the operative head is pressed on to the head port 12 for attaching thereto.

The head port 12 further comprises a lubricant delivery port 16, which may act as main or auxiliary liquid delivery port, and configured for delivering a lubricant or other generally-liquid substance therethrough. The lubricant or generally-liquid substance may be a simple lubricant, sexually stimulating substance including one or more active materials such as THC, CBD or selected combination between them. The lubricant delivery port 16 can be connected to a lubricant source to deliver the lubricant. The lubricant delivery port 16 is configured, i.e., structured, positioned, and oriented so as to connect to a lubricant port extension element of the operative head when the operative head is attached to the head port 12. The connection between the lubricant delivery port 16 and the lubricant port extension element is preferably leak-proof connection for delivery of the lubricant therethrough. Moreover, this connection is established during attachment of the operative head to the head port 12, i.e., by the single attaching action. Thus, the single action of attaching the operative head to the head port causes the attachment of the operative head to the head port, operational coupling of the motor 14 with the operational unit of the operative head, and connection between the lubricant delivery port 16 and the lubricant port extension element of the operative head.

In the illustrated example, the adult toy 10 further comprises a main head 20 attached to a main port 22. It is to be understood herein that although in the illustrated example, the main head 20 is fixedly attached to the main port 22, in other examples, the main port 22 can be configured for removably attaching thereto one or more main heads one by one. The replaceable main heads can have different shapes (curvatures), textures (dotted, striped, etc.), dimensions (for example, of insertable portion), and/or operational units. The main head 20 is configured for being inserted into a body cavity such as a vagina or anal cavity, of a user of the adult toy. The main head 20 is attached to the main port 22 along its longitudinal axis MLA, which also constitutes the main longitudinal axis of the adult toy 10. Although in the illustrate example, the main head 20 extends along the main longitudinal axis MLA of the adult toy 10, it is to be understood herein that in some examples the main head can extend at an angle with respect to the main longitudinal axis.

The main operational unit can be a vibrator unit and the adult toy 10 includes a main motor 24 configured to operate at least one moving member of the vibrator unit. In other words, the main motor 24 is configured to move the main head (moving member) in a predetermined pattern to cause stimulations at or inside the vagina when the adult toy is in use.

The adult toy further comprises a main lubricant delivery port 26, generally acting as main liquid/lubricant delivery port, configured for delivering therethrough a generally-liquid substance such as lubricant and/or active material to the vagina of the user. Typically, main lubricant delivery port 26 may be as or different from the lubricant delivered by the lubricant delivery port 16. However, in some embodiments, the adult toy may include a main liquid delivery port 26 for dispensing the generally-liquid substance within the user body cavity, and auxiliary delivery port 16 for dispensing the generally liquid substance at an auxiliary position that may be external to the suer's body cavity. The main head 20 includes holes 21 corresponding the main lubricant delivery port 26 so that the lubricant can be delivered through the main head into the vagina when the adult toy 10 is in use.

Although in all the examples of the adult toy 10 described herein, the adult toy 10 comprises the head port 12 as well as main head port 22, it is to be understood that the adult toy 10 can have one head port, i.e., head port 12 that is configured for removably and selectively attaching thereto one or more operative heads for performing corresponding functions with the sexual organ of the user. All the components corresponding the main head 20, for example, the main port 22, the main motor 24, the main lubricant delivery port 26, are to be understood as not being essential, and the adult toy 10 can be realized without these components with only the head port 12 and its corresponding components.

Generally, the adult toy exemplified herein also includes at least one sensor 23, located within the main head 20. Generally, the at least one sensor 23 is position at a location configured to be inserted into the user's body cavity to obtain sensing data from within the user's body cavity. Typically, the adult toy may include an arrangement of sensors 23, including one or more pressure sensors, temperature sensor, conductivity sensor, optical heart rate sensor, and optionally additional sensors. The arrangement of sensors are operable by the controller 60 mentioned further below, and configured to collect sensing data indicative of at least one physiological and environmental parameters of the adult toy. For example, the use of one or more conductivity sensors may provide sensing data indicative of level of electrical conductivity within the user's body cavity. This type of sensing data is processed by the controller to determine level of moisture within the body cavity, and may indicate a need for additional lubricant. Additionally, the use of one or more pressure sensors, located in selected locations along the main head 20, provides pressure variation data that can indicate muscle contractions that may be associated with sexual stimulation. Similarly, heart rate data can be obtained using pressure and/or optical sensors. Heart rate variation may also be used as indication of sexual stimulation level. The use of temperature sensor may provide data indicative of main head 20 location, i.e., being within the user's body cavity or outside thereof, and may provide temperature variations that can also indicate data on sexual stimulation. The use of sensor arrangement and collecting physiological data may also be used for periodic monitoring of the user's physiology and physical condition in long term use. The adult toy may utilize collection of physiological sensing data and store such data (locally) at the controller or associated electronic device, and/or remotely at a selected remote server, and provide the user with periodic analysis based on the collected sensing data.

As in the illustrated example, the adult toy 10 comprises the main head 20 attached to the main head port 22, the head port 12 constitutes an auxiliary head port 12 and the operative heads and their operational units constitute auxiliary heads and auxiliary operational units. Similarly, the longitudinal axis of the auxiliary head port 12 constitute an auxiliary longitudinal axis ALA, the motor 14 and the lubricant delivery port 16 constitute respectively an auxiliary motor 14 and an auxiliary lubricant delivery port 16.

The auxiliary longitudinal axis ALA is inclined at an acute angle A1 with respect to the main longitudinal axis MLA when seen from the side of the main head port 22 and the auxiliary head port 12. The angle A1 is selected for the adult toy 10 to be used such the main head 20 can be inserted into the vagina of the user and the auxiliary heads, when attached to the auxiliary port 12, can interact with the clitoris of the user. Generally, the main head may be inserted into body cavity of the user while auxiliary head attached to the auxiliary port may stimulate the user externally. In some examples, the connection between the auxiliary head port 12 and the main head port 22 can be made flexible enough to be adjusted by and according to the user while being rigid at least enough for the use of the adult toy 10. Thus, the adult toy 10 is configured to stimulate the vagina and the clitoris of the user simultaneously thereby significantly increasing the pleasure and/or relief whatsoever is the adult toy used for by the user. At the same time, the adult toy 10 is configured to selectively deliver lubricant (generally-liquid substance) that may include active materials such as THC and/or CBD through one or more of the delivery ports being inside the body cavity and/or at selected one or more external locations, e.g., inside the vagina and at the clitoris. The lubricant can be a sexually stimulating substance and/or can contain substances for medicinal use such as to address various sex related problems, as described in detail later herein below.

As can be seen in FIGS. 1C to 1E, the adult toy 10 comprises a lubricant inlet port 30 configured to be connected to a lubricant source containing a lubricant to be delivered through the adult toy 10. The adult toy 10 further comprises a main conduit 32 configured to deliver the generally-liquid substance (lubricant) from the lubricant inlet port 30 through the main lubricant delivery port 26, and an auxiliary conduit 34 configured to deliver said lubricant from the lubricant inlet port through the auxiliary lubricant delivery port 16, which is distant from the main liquid/lubricant delivery port 26. The adult toy further comprises a pumping mechanism 40 for pumping the lubricant from the lubricant inlet port 30 through each of the main conduit 32 and auxiliary conduit 34.

It is to be understood herein that although the adult toy 10 has been illustrated as having the features relating to the replaceability of operative heads as well as the lubricant being deliverable from two distant ports of the adult toy, the adult toy can be realized with any one set of these features. The adult toy 10 can be realized with only the features related to lubricant being deliverable from two distant ports of the adult toy without the adult toy necessarily having features related to replaceability of the operative heads, and vice versa. In other words, the presently disclosed subject matter includes within its scope an adult toy having the features related to the lubricant being deliverable from two distant ports of the adult toy with fixedly attached operative heads and the main head. For instance, the adult toy 10, according to some examples, can comprise fixed main head 20 and a fixed auxiliary head at the auxiliary head port 12, each having respective lubricant delivery ports configured for delivering the lubricant inside the vagina (through the main head) and at the clitoris (through the auxiliary head).

In the illustrated example, the main conduit 32 connects the lubricant inlet port 30 to the main lubricant delivery port 26, and the auxiliary conduit 34 connects the lubricant inlet port 30 to the auxiliary lubricant delivery port 16. The conduits 32 and 34 transfers the lubricant from the lubricant inlet port to the lubricant delivery ports for the delivery of the lubricant to the user therethrough. The lubricant inlet port is configured to be connected, to establish a fluid communication, to a lubricant source. The lubricant source can be any internal or external source containing the lubricant that is to be delivered to the sexual organ of the user. The lubricant can be a sexually stimulating substance and/or a medicinal drug to address sex related problems. In the illustrated example, the lubricant source is a replaceable lubricant capsule that can be received within the adult toy. However, in other examples, the lubricant source can be a refillable cartridge or an external single or multiple use lubricant source. In some examples, the lubricant source can include more than one lubricant sources, each containing different lubricants to be delivered to the vagina and the clitoris of the user. In such examples, the lubricant inlet port can be configured to be connected to the multiple lubricant sources and deliver one lubricant though the main conduit and another lubricant through the auxiliary conduit.

In the illustrated example, the lubricant source can be a single lubricant source, i.e., the lubricant capsule 50. The lubricant inlet port 30 is a double one-way valve configured to be fluidly connected to the lubricant capsule 50 at one end and the main and the auxiliary conduits 32 and 34 on the other side. The pumping mechanism 40 comprises a first peristaltic pump 42 associated with the main conduit 32 and configured to pump the lubricant from the lubricant source 50 and the lubricant inlet port 30 through the main conduit 32. The pumping mechanism 40 further comprises a second peristaltic pump 44 associated with the auxiliary conduit 34 and configured to pump the lubricant from the lubricant source 50 and the lubricant inlet port 30 through the auxiliary conduit 34.

The main conduit 32 extends through the main head port 22 into the main head 20 and is in fluid communication with the main lubricant delivery port 26. The first peristaltic pump 42 is configured to deliver the lubricant through the main conduit 32 and the main lubricant delivery port 26 inside the vagina of the user via the main head 20 when the main head 20 is inserted in the vagina. The second peristaltic pump 44 is configured to, simultaneously or independently of the first peristaltic pump 42, deliver the lubricant through the auxiliary conduit 34 and the auxiliary lubricant delivery port 16 at the clitoris of the user via the auxiliary head when the auxiliary head is interacting with the clitoris. In order to deliver the lubricant to the clitoris, the auxiliary lubricant delivery port 16 is configured for being connected to the lubricant port extension element of the auxiliary head when the auxiliary head is attached to the auxiliary head port 12.

The adult toy 10 further comprises a controller 60 configured for controlling the operations of the adult toy 10. The controller 60 is configured to receive user preference data as user input, and sensing data from the one or more sensors 23, and to utilize the received data for determining operation of the main and/or auxiliary motors 14 and 24 and/or the pumping mechanism 40, i.e., the first peristaltic pump 42 and the second peristaltic pump 44. Operation of the controller 60 is generally described with reference to FIGS. 2 and 3 above and is described in more detail further below. The controller can control the first and second peristaltic pumps either in coordination with each other or independently of each other. While operating the peristaltic pumps 42 and 44 in coordination with each other, the controller can control the operation of the peristaltic pumps 42 and 44 according to a predetermined pattern to suit the requirement of the user.

Further, the controller 60 is configured to control the operation of the auxiliary motor 14 and/or the main motor 24, either independently of or in coordination with each other according to a predetermined pattern. In some examples, the controller 60 can be configured to control the operations of the auxiliary motor 14, the main motor 24, the first peristaltic pump 42, and the second peristaltic pump 44 according to one or more predetermined pattern, each one to suit a particular requirement or preference for the sexual stimulation to be achieved by the adult toy. In some examples, the adult toy 10 can comprise different controllers (separate or forming a part of a single control system) for controlling the operations of the different components.

As can be seen in FIGS. 1F to 1H, the adult toy 10 comprises a capsule receiving portion 52 configured to receive the lubricant capsule 50 therewithin and a locking mechanism 70 configured to lock the lubricant capsule 50 within the cartridge receiving portion 52 by a locking action. The lubricant inlet port 30 is configured to be in fluid communication with the lubricant capsule 50 when the lubricant capsule 50 is positioned within the capsule receiving portion 52. The inlet port 30, the capsule receiving portion 52, and the locking mechanism 70 are configured such that the fluid communication between the lubricant capsule 50 and the lubricant inlet port is established during said locking action of the locking mechanism 70. The lubricant inlet port 30 and the locking mechanism 70 are so positioned, and even dimensioned and oriented, that the locking action causes the engagement of the lubricant inlet port 30 with the lubricant capsule 50 for establishing said fluid communication.

It is to be understood herein that although the adult toy 10 has been illustrated as having the features relating to the replaceability of operative heads, features relating to the lubricant being deliverable from two distant ports of the adult toy as well as the features relating to the establishment of fluid communication between the lubricant inlet port and the lubricant capsule during the locking action, the adult toy can be realized with any one set of these features. The adult toy 10 can be realized with only the features related to the establishment of fluid communication between the lubricant inlet port and the lubricant capsule during the locking action without the adult toy necessarily having the features related to the lubricant being deliverable from two distant ports of the adult toy and the features related to replaceability of the operative heads. In other words, the presently disclosed subject matter includes within its scope an adult toy having the features related to the establishment of fluid communication between the lubricant inlet port and the lubricant capsule during the locking action with replaceable or fixedly attached operative heads and the main head, or even with a single head, and/or even without the features related to the lubricant being deliverable from two distant ports. For instance, the adult toy 10, according to some examples, can comprise a single fixed or replaceable head including the features related to the establishment of fluid communication between the lubricant inlet port and the lubricant capsule during the locking action.

In the illustrated example, the adult toy 10 comprises a connector section 54 to which the capsule receiving portion 52 is removably connected via the locking mechanism 70. The lubricant inlet port 30 and a first locking part 72 of the locking mechanism 70 are positioned at the connector section 54. A second part 74 of the locking mechanism is positioned at the cartridge receiving portion 52. When the capsule receiving portion 52, having the lubricant capsule 50 positioned therewithin, is connected to the connector section 54, the lubricant inlet port 30 engages the lubricant capsule 50 and the fluid communication is established, i.e., by the locking action between the first and the second parts 72 and 74 of the locking mechanism 70. The lubricant capsule 50 includes a pierceable portion 51 and is positioned in the capsule receiving portion such that the pierceable portion 51 faces the lubricant inlet port 30. The lubricant inlet port 30 has a conical end 31 configured to pierce the pierceable portion 51 when the lubricant capsule 50 is pressed thereon.

Although in the illustrated embodiment, the locking mechanism 70 is a bayonet lock, it is to be understood herein that the locking mechanism can be any other locking mechanism such as a snap fit lock or a press lock by virtue of dimensions of the cartridge receiving portion and the connector section.

As can be seen in FIGS. 1A to 1H, the adult toy 10 further comprises a heating mechanism 80 configured to heat the lubricant in the lubricant capsule 50 before the lubricant is delivered to the user organs. It is to be understood herein that the lubricant can be delivered from any other lubricant source than the capsule 50. The lubricant is a cannabinoid extract and can include CBD oil or THC oil. The heating mechanism may also be connected to the controller 60 for selective operations in accordance with operational instructions from the controller. For example, the controller may operate the heating mechanism based in user preference data indicative of preferred temperature difference between the user's body temperature and temperature of the lubricant/generally-liquid substance. To this end, the controller 60 may also utilize temperature data collected by the one or more sensors 23 for determining body temperature of the user, and variations thereof during operation.

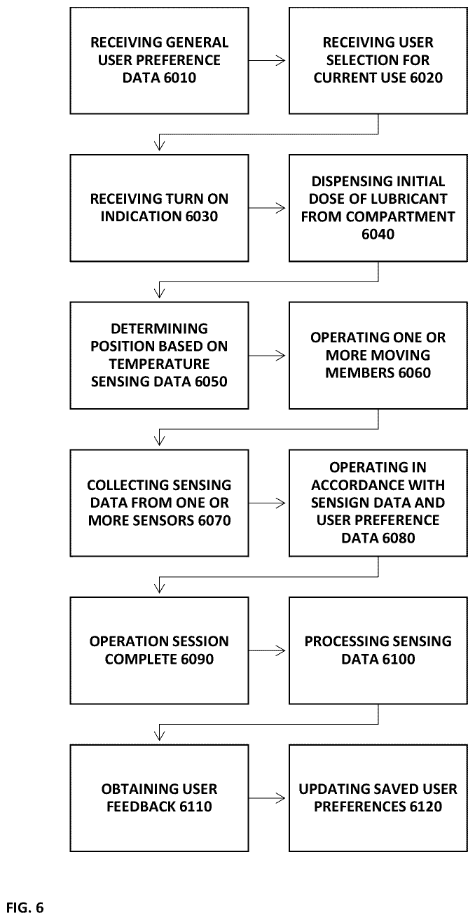

As indicated above, the adult toy utilizes a controller marked as 110 in FIGS. 1 and 2 or 60 in FIGS. 4B, and one or more sensors marked as 23 in FIGS. 4A and 4B. The controller may generally include a processor and memory circuitry (PMC) and may be operable for determining operation of the adult toy in accordance with user preference data and sensing data. Typically, the controller may be configured to communicate with an external electronic device for obtaining user instructions, perseverance data, and optionally also for providing the user with output data and/or request for further input. In this connection, FIG. 5 exemplifies an electronic device 500, e.g., smartphone, and some user input/output fields determining or used in operation of the adult toy according to some embodiments of the present disclosure. It should be noted that the input/output data fields exemplified in FIG. 5 indicate certain activities/information fields and may or may not be presented at any screen provided for operating of the adult toy.

As exemplified in FIG. 5, at operation of the adult toy, a user may be asked to provide one or more of certain user data 510, general operation preferences 520, and additional information to determine operation schemes of the adult toy. The user may be asked to fill out a questionnaire indicating user data 510 and general preferences 520, and may be directed to one or more screens requesting for current preferences 525 that may include operation duration preferences 528. Additionally, during operation, the electronic device may be used to transmit user input 530 including, e.g., instruction for current operation scheme such as a request for vibration burst, variation of frequency or amplitude of operation of the one or more moving members, request for additional lubricant applied during operation, or change in amount of active generally-liquid substance being dispensed. Further, user controls may include one or more dials 535 enabling the user to selectively adjust operation of the adult toy and/or select certain operations at selected times. For example, the user may request dispensing additional generally-liquid substance (lubricant and/or active substance) through the main or auxiliary delivery ports at selected times, operate one or more of the main or auxiliary moving members in selected direction, vibration magnitude and/or frequency.

Additional data that may be collected from the user, e.g., using communication with the electronic device 500, may include user input following operation of the adult toy 540. Such user input may include indication of stimulation level 545, e.g., did the user reach orgasm during operation of the device or not. The controller of the adult toy may operate to store user preferences and user input in the memory thereof for adjusting its operation as described in more detail further below.

In some embodiments, the user may be able to select between a pre-made list of fixed operation programs 550. Such pre-prepared operation programs may include selection of vibration pattern of the moving member, use of one or more auxiliary moving members, selected auxiliary heads or main head.

Additional data field that may be available via the electronic device 500 according to some embodiments of the present disclosure include an indication 560 of type of the generally-liquid substance used by the adult toy device. Further, the electronic device may provide the user with physiological and operation data collected by the one or more sensors during device operation 570, and optionally certain analysis of the collected sensing data and toy operation. For example, the adult toy may store and provide statistical data on number of uses, length of each use session, level of measure sexual stimulation, user response to different stimulation patterns etc.

It should be notes that the user interface is described herein as operating within an external electronic device, the present disclosure may utilize various other formats of user interface where the user is capable of providing certain preference data to be stored at the controller of the adult toy.

Additionally, in accordance with some embodiments, the user input may include data on a first desired short operational duration or a second desired longer duration. There may be of course more than two inputs informative of corresponding different operational time durations. The time duration may be informative of the desired time duration of operation the adult sex toy until the user arrives to satisfaction (typically orgasm).

Further, additional user preferences may relate to additional functionalities of the adult toy. For example, user preferences on operation strength/magnitude and/or frequency such as “hard”, “soft” and/or possibly other; preferred lubrication levels such as high use of lubricant determining lubricant dispensing thresholds. Additionally, operation user preferences may relate to operation during and/or after sexual climax, for example a user may indicate increased operation magnitude and/or frequency operation in response to sensing data indicating sexual climax (e.g., learned data based on user experience, pattern of pressure variations, increased heart rate, increased level of humidity not related to lubricant dispensing and/or combination of two or more such indications).

The user preference input may be fed through physical means, say switch or through digital input such as using an associated electronic device as exemplified above through a screen residing locally or in remote device, all depending upon the particular application.

Reference is made to FIG. 6 exemplifying an operational scheme of an adult toy according to some embodiments of the present disclosure. As exemplified, the adult toy may be operated by the controller thereof, for receiving user general user preference data 6010 and user selection for current session 6020. Such user preference data may include general user data such as age, gender, sexual experience level, and potentially medical conditions, as well as user preference data relating to device operation. For example, user preference data may relate to data on preferred lubrication levels, vibration strength and/or frequency, location of preferred stimulation (e.g., vagina, anus, clitoris etc.), etc. The controller may operate to store the general user preferences and user data in the memory for additional operation sessions. The user's current preferences for a certain use may include any variation of the suer preferences and may also include data on use duration. This separation enables the device to learn user preferences, while allowing flexibility enabling the user to take different approach when desired. Generally, user preferences may also include an experimental indication, instructing the controller to add certain random variation to operation of the moving member, one or more pumps etc., in order to allow the user to experiment while not directly anticipating operation pattern of the adult toy.

To initiate a session, the controller is responsive to a “turn on” command from the user 6030, indicating that the suer is ready to use the adult toy. In response to initial “turn on” indication, the controller may operate for initial dispensing of lubricant/generally-liquid substance 6040. The controller is further operating for collecting sensing data using the one or more sensor, including for example temperature data. Using temperature data, the controller may determine if the main head of the adult toy is located within body cavity or external thereto 6050. Typically, the controller may determine location of the main head based on determining temperature within the range of 36° to 40° associated with body temperature, as opposed to lower temperatures measure externally to the user body cavity.

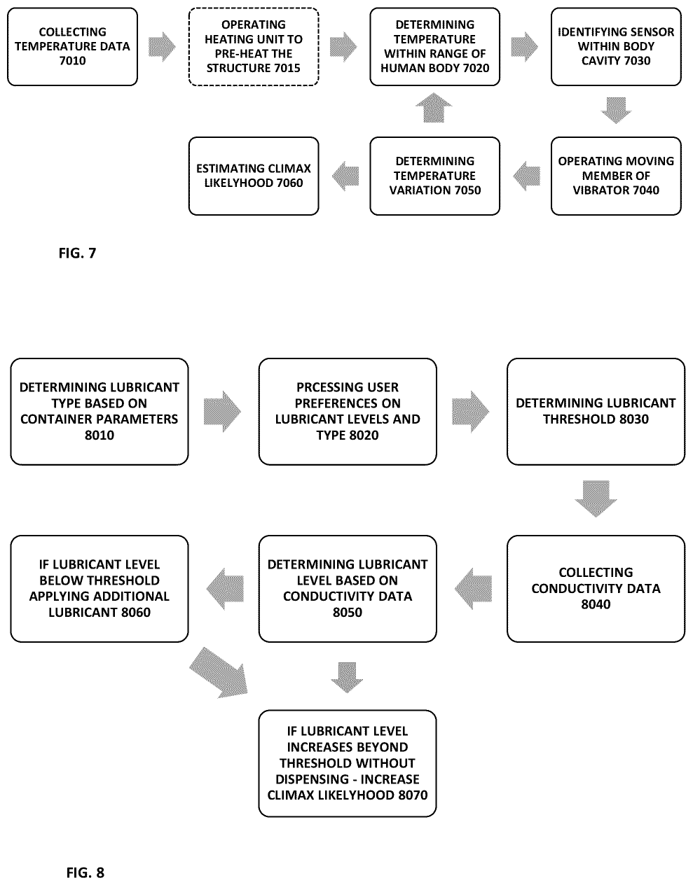

Based on user preference data, the controller may operate the one or more moving members 6060 in selected vibration magnitude, frequency and pattern. The controller may operate one or more moving members associated with main and/or auxiliary head for stimulating different body regions in accordance with suer preferences. During operation, the controller is operable for receiving sensing data from the one or more sensors 6070. As indicated herein, the sensing data may include data on conductivity, pressure variations, temperature variations, heart rate data etc. collected by the one or more sensors at a selected sampling rate. The controller may process the sensing data in accordance with the pre-provided user preference data and adjust operation of the vibrator accordingly 6080. For example, the controller may vary operation of the one or more motors causing vibrations of the moving members and/or operation of one or more pumps for dispensing the generally-liquid substance through main and/or auxiliary delivery ports, in accordance with sensing data as exemplified further below with references to FIGS. 7 to 9. During operation, the controller is typically operable for receiving and processing sensing data from the one or more sensors and adjust operation of the one or more motors and/or one or more pumps accordingly.

For example, the controller may receive and process sensing data from one or more conductivity sensors to determine moisture levels within the body cavity and/or at an external location where structure of the adult toy interfaces the user's body (e.g., clitoris). Based on variation in conductivity levels, the controller may determine if additional amount of generally-liquid substance or lubricant is required and apply the one or more pumps to dispense the substance at the respective location via the main liquid delivery port and/or auxiliary liquid delivery port. A threshold for conductivity levels and/or amount of generally-liquid substance to be applied may be determined using an initial parameter adjusted by user preference data. Further, such threshold may be updated between use sessions based on user feedback and recorded sensing data.

Additionally, the controller may operate for receiving and processing sensing data from one or more of conductivity sensors, pressure sensors, and heart rate sensors, for determining level of sexual stimulation of the user. For example, in response to a certain combination increase in heart rate, increase in conductivity (not related to pump activity) and pattern of pressure variations, the controller may determine level of sexual stimulation and identify climax. Further, the controller may utilize pre-stored user preferences associated with operation preferences during climax and apply such preferences in response to determining sexual stimulation level above a respective threshold. For example, a user may indicate in user preferences, that the adult toy enhances vibration strength or frequency during climax for a selected period. Additionally, or alternatively, a user may indicate operation of auxiliary moving member for external stimulation close to climax, and/or dispensing additional amounts of generally-liquid substance. Additional user profiles may be pre-stored in accordance with user provided data. Further, the controller may store sensing data and operational data during each operation session and may operate for processing such stored data for optimizing future operation sessions to user satisfaction. Such processing may utilize machine learning and may be performed locally at the controller or at a remote server by direct communication or via the respective electronic device.

The user preferences, and operation of the adult toy may also include data on a desired operation period. Accordingly, the controller may operate using stimulation pattern for directing the user to preferred stimulation level, and preferably up to climax within the desired operation period. To this end, the controller may record and store user reactions to various stimulation types/patterns and utilize local or remote processing of the suer reactions to identify preferred patterns enhancing the suer's stimulation level.

To this end, following end of an operation sessions 6090, the adult toy may operate for processing the collected sensing data 6100. Processing the sensing data may be performed locally or include transmitting the collected sensing data to a remove processing utility. The processing may utilize machine learning techniques for identifying patterns ion user response to various stimulation patterns. The adult toy may also generate a session end notification to the user and be responsive to input data from the user 6110. User feedback may include user indication on pleasure levels during operation, stimulation patterns that are pleasurable or less pleasurable, confirmation or disconfirmation on climax, etc. As indicated above, the controller may utilize the sensing data, before and/or after processing thereof, and user feedback data for updating user preferences and storing the updated user preferences in the memory for later use 6120.

As indicated above, the adult toy of the present disclosure may utilize wired or wireless communication to one or more associated electronic devices that may provide communication port to a network enabling access to one or more remote servers for processing, and/or enable simple and direct user interface. Accordingly processing of the sensing data in accordance with user preferences and/or user feedback data may be performed locally at the PMC and/or remotely at a dedicated server unit, while maintaining data privacy for the user data, and utilizing anonymized data.

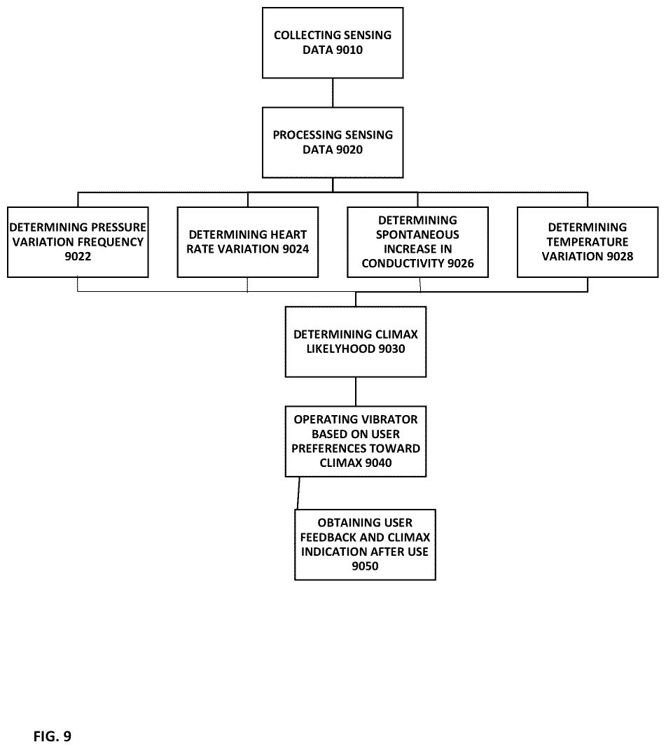

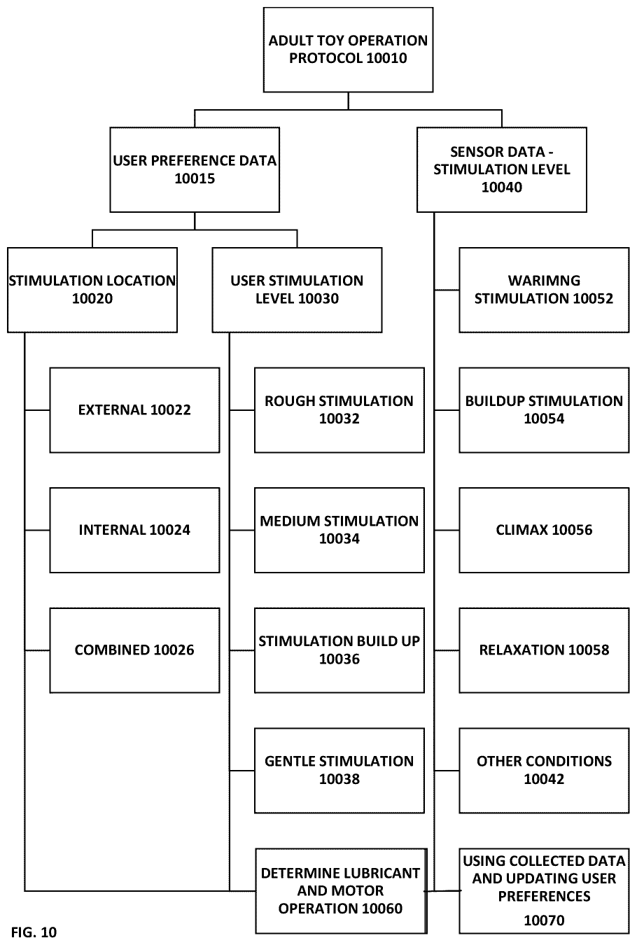

In this connection, reference is made to FIGS. 7 to 9 exemplifying certain operation actions of the adult toy and controller thereof according to some embodiments of the present disclosure. FIG. 7 relates to temperature data; FIG. 8 relates to conductivity and lubrication data sensing and FIG. 9 relate to determining climax likelihood of a user.

As shown in FIG. 7, the controller generally operable for collecting temperature data 7010 from one or more temperature sensors of the adult toy. The temperature sensors may be any type of temperature/heat sensors including for example thermocouple sensors, generally including at least one sensor located at main head of the adult toy at a position expected to be inserted into user's body cavity. The controller may operate a heating unit to pre-heat the structure and/or generally-liquid substance to preferred operating temperature in accordance with user preferences 7015. The controller utilized the temperature data to determine temperature variations and determine if temperature is within range associated with human body temperature 7020, e.g., 36° C. to 40° C. Accordingly, the controller may determine that the respective one or more sensors are within the user's body cavity 7030 thereby indicating status and position of the adult toy. In accordance with user preferences and operation plan, the controller may operate one or more moving members 7040 to provide sexual stimulation. During operation, the controller further monitors and determines temperature variations 7050, and may relate to increased temperature variation as a signal for estimating climax likelihood 7060. As indicated above ad described in more detail below, the controller may request user indication and confirmation on climax to update its estimation.

FIG. 8 exemplifies conductivity and humidity sensing and related processing. As shown, the controller may utilize pre-provided data, or data provided within generally-liquid substance capsule for determining lubricant type 8010. Lubricant type may relate to level of conductivity of the lubricant as well as to existence of one or more active materials such as THC, CBD etc., that may be provided within the lubricant for additional sexual stimulation. The controller further processes the pre-stored user preferences on lubrication levels 8020 and type of lubrication for determining conductivity/lubrication threshold 8030. During operation, the controller operates the one or more conductivity sensors for collecting conductivity data 8040. To this end, the conductivity sensors may generally include at least one sensor located at main head, and may include one or more additional sensors on auxiliary head, providing conductivity sensors at locations on interface between adult toy head and user's body. The controller utilizes conductivity data, and data on type of the generally-liquid substance (lubricant) for determining lubricant level based on the conductivity data 8050. This processing may include pre-provided data on the generally-liquid substance, as well as user data relating to conductivity of body fluids of the user. If lubricant level is determined to be below the determined conductivity/lubrication threshold, the controller may operate the one or more pumps for dispensing additional generally-liquid substance (lubricant) 8060 via the respective liquid delivery port to bring lubricant levels to desired levels. Additionally, the controller may utilize conductivity data for determining climax likelihood 8070. In this connection, if conductivity measures increase without dispensing of additional generally-liquid substance, it may be assumed that the user's body releases body fluids as a result of sexual stimulation. Accordingly, the controller may determine increased climax likelihood, and may also request user indication/confirmation after operation session.

The example of FIG. 9 illustrates the sue of additional sensing data for determining climax likelihood according to some embodiments of the present disclosure. As shown, the controller may operate for collecting sensing data 9010 including e.g., heart rate, conductivity, pressure, and temperature, and may include one or more additional sensors. The controller, or its respective processor (PMC) operates for processing the sensing data 9020 in accordance with pre-stored user preferences to determine variations and patterns in the collected sensing data. Typically, the controller may correlate variations and patterns collected using the different sensor types. For example, the controller may operate for determining pattern and amplitude of pressure variations 9022 collected by the pressure sensors; determine heart rate variations 9024 collected by the heart rate sensor; determine data on increase of conductivity 9026, typically being irrespective of dispensing additional generally-liquid substance thus indicating spontaneous release of body fluids; and determine temperature variations associated with increase of body temperature due to sexual stimulation 9028. The controller may operate to determine climax likelihood 9030 based on each one of the sensing data types by itself. However, in some preferred embodiments, the controller utilizes correlations between variation of different physiological conditions for increasing accuracy of determining climax likelihood. Accordingly, the controller may utilize combination of one or more of increased frequency of pressure variations, combined with heart rate increase, release of body fluids and increase of temperature, to improve determining of climax likelihood of the user. In this connection, determining climax likelihood may relate to determining if the user is approaching sexual climax, and or is reaching sexual climax. Accordingly, processing the sensing data may be used for identifying level of sexual stimulation and determining operation patterns of the vibrator (adult toy) according to the user preferences and sensing data to direct the suer toward climax 9040, and determining operation during climax in accordance with suer preferences at this stage.

Further, as indicated above, the adult toy of the present disclosure and its respective controller may be configured for obtaining further user feedback 9050 to enable updating of user preferences data and enhance accuracy and selectivity of processing of sensing data. In this connection, the user input may be voluntary, and may relate to any selected detail level agreed by the user. The suer may agree to provide indication of pleasurable over less pleasurable, provide marking throughout the operation sessions on user experience at each point in time, relate to specific operation patterns etc., while the controller may utilize user's feedback for labeling collected sensing data and utilize location or remote processing for analyzing the sensing data with respect to suer feedback and updating user preferences accordingly.

FIG. 10 further exemplifies a list of decision parameters relating to operation of the adult toy according to some embodiments of the present disclosure. Typically, the adult toy may include an initial, basic, operation protocol 10010, which may generally be adjusted to user preferences 10015 typically provided by the user as input. The user preferences may relate to general preferences of the suer, as well as current preferences that apply for each specific operation session. Accordingly, operation of the adult toy may be determined based on the user preferences data 10015, combined with detected stimulation data on stimulation level, 10040, typically obtained from the one or more sensors.

For example, the suer preference data 10015 may include data on stimulation location 10020 and on stimulation level preferred by the user 10030. In this connection, a user may prefer external stimulation 10022, e.g., using an auxiliary head stimulating external body parts, internal stimulation 10024 where the adult toy is mostly active within body cavity, or a combined stimulation 10026, being both internal and external. Additionally, the user preferences may relate to strength of stimulation 10030, running between rough 10032, medium 10034, buildup of the stimulation from gentle to rough 10036 or gentle stimulation 10038. It should be noted that the number of options and specific strength gauge for each option may vary and are not bound by this specific example.

During operation, based on the suer preferences data, the controller of the adult toy according to the present disclosure receives and processes sensing data from the one or more sensors, e.g., including one or more of conductivity (humidity) sensor, pressure sensor, temperature sensor and heart rate sensor, to determine user response to stimulation and stimulation level 10040. For example, the controller may determine user status being between warmup level 10052, buildup stimulation 10054, reaching climax 10056, and post climax relaxation 10058. Processing of the sensing data may also determine other conditions 10042, which may relate to other user conditions such as medical conditions, additional user input, stopping of the session by the user etc.

The controller may utilize a mathematical algorithm, machine learning, or other processing technique for processing the input data and determine current operation of motor and/or lubricant dispensing 10060. In this connection, following user preferences, the controller may determine a level αᵢ of motor operation, for each motor (i) operating in the adult toy. This may include one or more main head motors and/or one or more auxiliary head motors. Additionally, the controller may determine one or more lubricant thresholds βⱼ relating to lubricant (generally-liquid substance) dispensing via the main and/or auxiliary conduits. In some embodiments, the lubricant thresholds and motor operation levels may also be determined based on data lubricant material. For example, lubricant containing active materials such as CBD, THC or other active materials may be dispensed at selected amounts while the actual amounts and timing may vary based on the active material.

During, or following a use session, the controller may operate to update the pre-stored user preference data with data on user response to stimulations 10070. In this connection, the controller may assign stimulation scores to different modes of operation, at respective stimulation levels of the user. For example, motor operation at a certain frequency may be assigned with one score when used for warmup stimulation, another score when used during buildup of the sexual stimulation, a third score when used during orgasm, and a fourth score when used during relaxation. The controller may determine user response to different actions of the adult toy at the respective stimulation stages and raise of reduce the respective score in accordance with user response as collected in the sensing data. For example, a response associated with gradual increase of heart rate during warmup or buildup stimulation may cause increase of the score. This is while sharp raise is heart rate, possibly combined with pressure and temperature reduction, may indicate the user rejection of the toy operation, leading to reducing of the respective score of this operation.

Further, in some embodiments, the adult toy may register mood data of the user. Such mood data may be provided by the user in user reference data prior to operation session. The controller may store data on mood levels and respective sensing data collected at initial stages of the respective use sessions. Further, the controller may operate to correlate general sensing data at early use stages to previous data collected with indicated mood data provided by the user. The controller may further utilize such correlation for estimating mood state of the user in further use and adjust operation levels accordingly.

Starting from a basic operation level, based on preferences and optionally mood data selected by the user. The controller may vary the operation level based on input on stimulation level obtained by the one or more sensors in order to provide the user with optimal experience within preferred operation duration (typically determined by the user). For example, increase in heart rate may indicate progressing stimulation level, as a result the controller may raise operation level (amplitude and/or frequency of vibrations) by selected amount. This is while increase in heart rate over certain rate, may indicate over stimulation or other conditions, leading to the controller reducing operation level to provide the suer with preferred buildup/warmup period. Similarly, increase in temperature, and/or pressure variations may indicate buildup in stimulation level. Accordingly, the controller may determine if the buildup rate is within user preferences and increase/decrease of maintain operation level.

Further, the controller may determine that stimulation level is reaching climax 10056 based on a combination of one or more of increase in heart rate, pressure variation pattern, temperature increase, and spontaneous (unrelated to lubricant dispensing) increase in humidity. The controller may store user preferences for climax operation, which may include increase operation level, maintaining operation level, or gradual decrease in operation level, and operate the one or more motors accordingly to bring the suer to relaxation with selected climax duration.

For example, the controller may determine climax based on identifying one or more of the following: pelvic muscles contraction at a rate of about 1 Hz within a duration of at least 5 seconds; enhanced humidity that is not correlated with lubricant dispensing; heart rate increase by about 10-25% of warmup heart rate; and temperature increase by 0.5° C. to 1° C. or more during operation. The climax period may take between 20-35 seconds; however, this period may vary between users.

Additionally, the controller may adjust heating of the structure head and/or generally-liquid substance in accordance with user preferences and may adjust heating level to climax level to equalizes temperatures and enhance pleasure.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the presently disclosed subject matter may be, at least partly, implemented on a suitably programmed computer. Likewise, the presently disclosed subject matter contemplates a computer program being readable by a computer for executing the method of the presently disclosed subject matter. The presently disclosed subject matter further contemplates a non-transitory computer-readable memory tangibly embodying a program of instructions executable by the computer for executing the method of the presently disclosed subject matter.

Those skilled in the art will readily appreciate that various modifications and changes can be applied to the embodiments of the presently disclosed subject matter as hereinbefore described without departing from its scope, defined in and by the appended claims.

1. An adult sex toy comprising:
a structure comprising a main head configured to be inserted into a user's cavity;
at least one moving member configured to generate selected vibration movement of said structure;
at least one sensor located in said main head of said structure and operable for generating sensing data indicative of at least one parameter collected from the user's body cavity; and
a controller connectable to said at least one sensor, said controller is configured and operable for receiving and processing said sensing data and for selectively operating said at least one moving member in accordance with one or more conditions determined based on said sensing data.

2. The adult sex toy of claim 1, further comprising a compartment having an inner volume and adapted to contain with a selected generally-liquid substance, a main conduit configured to deliver said generally-liquid substance from said compartment through a main liquid delivery port, and at least one pump adapted for pumping said generally liquid substance through said main conduit, said controller is further configured to operate said at least one pump for delivering said generally-liquid substance through said main liquid delivery port in accordance with one or more conditions determined based on said sensing data.

3. The adult sex toy of claim 2, further comprising at least one auxiliary conduit configured to deliver said generally liquid substance from the compartment through an auxiliary delivery port distant from the main liquid delivery port; said at least one pump is further adapted for pumping said generally liquid substance through said auxiliary conduit and through said main conduit.

4. The adult sex toy of claim 2, wherein the at least one pump comprises a first peristaltic pump configured to pump the generally liquid substance from said compartment through the main conduit, and a second peristatic pump configured to pump the generally liquid substance from said compartment through the auxiliary conduit.

5. (canceled)

6. (canceled)

7. (canceled)

8. The adult sex toy of claim 1, wherein said moving member further comprising at least one motor, said controller is further connectable to said motor for selectively controlling one or more parameters of operation of said at least one motor in accordance with said sensing data.

9. The adult sex toy of claim 1, wherein said controller comprises at least one processor and memory and is configured to received and store input data indicative of one or more predefined user preference modes; said controller is further configured for processing said sensing data in accordance with said one or more user preference modes.

10. The adult sex toy of claim 1, wherein said at least one sensor comprises at least one pressure sensor positioned near top end of said moving member and one or more additional pressure sensors located at one or more positions along said moving member, said at least one pressure sensor and one or more additional pressure sensors are operable for providing sensing data indicative of time-varying pressure applied thereon, said data on time-varying pressure being indicative of use and user stimulation level and said sensing data comprises data indicative of time-varying pressure along said moving member.

11. (canceled)

12. The adult sex toy of claim 1, wherein said at least one sensor comprises at least one temperature sensor positioned near top end of said moving member, said sensing data comprises temperature variation data.

13. The adult sex toy of claim 12, further comprising at least one additional temperature sensor positioned at distal end of said moving member, such that said additional temperature sensor is external to said user's body cavity when said moving member is inserted into the user's body cavity, said additional temperature sensor provides sensing data indicative of external temperature, said sensing data thereby comprises data indicative of temperature difference between said at least one temperature sensor and said at least one additional temperature sensor.

14. The adult sex toy of claim 1, wherein said controller is configured for processing said sensing data to determine temperature difference in data obtained from said at least one temperature sensor and said at least one additional temperature sensor, said controller is configured to use data on said temperature difference and determine data on presence of said moving member within said user's body cavity; and wherein said controller is configured to determine environment status indicative of presence of said moving member within said user's body cavity based on temperature data collected by the at least one sensor.

15. (canceled)

16. The adult sex toy of claim 1, wherein said at least one sensor comprises at least one conductivity sensor, said controller is configured for processing conductivity data ins aid sensing data to determining one or more parameters comprising at least data on liquid material composition around said moving member.

17. The adult sex toy of claim 1, wherein said controller is configured for processing said sensing data to determining data indicative of heart rate of a user based on periodic variations in said sensing data in the range of 50 to 200 beats per minute, and for utilizing said heart rate data to determine estimated stimulation level of the user.

18. The adult sex toy of claim 1, wherein the controller comprises pre-stored user preference data, and wherein said controller is configured and operable for receiving and processing sensing data from the at least one sensor in accordance with user preference data and determining user stimulation level during operation; and wherein said controller is configured and operable for correlating sensing data collected from one or more sensors collecting respectively one or more different types of sensing data for determining user stimulation level during operation.

19. (canceled)

20. (canceled)

21. (canceled)

22. (canceled)

23. The adult sex toy of claim 1, wherein said generally-liquid substance comprises one or more active elements comprising THC or CBD, and wherein said controller is configured to receive input data indicative of presence of one or more active elements in said generally liquid substance and to selectively operate in at least first or second operation modes selected in accordance with presence of active elements in said generally liquid substance.

24. (canceled)

25. The adult sex toy of claim 1, wherein said controller comprises stored data indicative of one or more operational programs preselected by a user, and for processing said sensing data to determine one or more operational parameters of said at least one moving member and said at least one pump in accordance with a selected operational program in said stored data.

26. The adult sex toy of claim 25, wherein said one or more operational programs comprise data on at least first short duration and a second longer duration, and wherein in response to selection of said second duration said controller is configured to perform repeatedly:
determining if, based on said sensing data, the stimulation intensity will lead to satisfaction faster or slower than said desired duration and in response thereto modify at least one of (i) a moving command to modify at least one of moving magnitude and speed of said moving member; (ii) a dispending command to modify the released dosage of said generally liquid substance from the compartment.

27. (canceled)

28. (canceled)

29. (canceled)

30. A method for controlling a battery operated adult sex toy configured according to claim 1, the method comprising:
(a) using at least one pre stored user preference data comprising at least data indicative of desired duration of operation of the toy until user's sexual stimulation arrives to satisfaction;
(b) obtaining, from said at least one sensor, sensing data indicative of one or more physiologic originated signals from the user's body; said physiologic originated signals being informative of the sexually stimulation intensity of the user while using the toy;
(c) processing sensing data to determine respective physiologic originated signals and determining at least one of: (i) a moving command for controlled modification of the magnitude of move of the moving member, and (ii) a dispensing command, for controlled dispensing of the stimulating substance from the cartridge by said dispensing control member,
thereby substantially matching the actual duration of operation of the toy with the desired input duration of operation, until satisfaction is arrived.

31. A method for operating an adult sex toy, the method comprising:
obtaining user preference data, and storing the suer preference data in a memory;
responding to user initiation instruction for operating the adult sex toy;
operating one or more moving members for stimulating one or more selected user body parts;
collecting sensing data comprising at least one of: pressure variations, heart rate, humidity, and temperature, from vicinity of the one or more selected user body parts;
varying operation of the one or more moving members in accordance with said sensing data and respective user preference data to bring the user to satisfaction;
updating stored user preferences based on sensing data for additional use sessions.

32. The method of claim 31, further comprising processing the sensing data and determining user stimulation level accordingly.

33. The method of claim 32, wherein said processing user stimulation level comprises determining a combination of time correlated of one or more of pressure variation frequency within range between 0.5 Hz and 2 Hz, heart rate increase within the range 10%-25% of initial heart rate, temperature increase between 0.5° C. and 3° C., and humidity increase that is not related to lubricant dispensing.
